(19) United States
(12) Patent Application Publication
(10) Pub. No.: US 2023/0270861 A1
(43) Pub. Date: Aug. 31, 2023

(54) Inhibitors of the Artemin Pathway for Treatment of Cancer

(71) Applicant: The University of Chicago, Chicago, IL (US)

(72) Inventors: Ralph WEICHSELBAUM, Chicago, IL (US); Hua Laura LIANG, Chicago, IL (US); Yuzhu HOU, Chicago, IL (US)

(21) Appl. No.: 18/006,482

(22) PCT Filed: Jul. 22, 2021
(86) PCT No.: PCT/US2021/042849

§ 371 Date: Jan. 23, 2023

Related U.S. Application Data
(60) Provisional application No. 63/055,284, filed on Jul. 22, 2020.

(51) Int. Cl. A61K 41/00; A61K 31/4995; C07K 16/28; A61N 5/10; C12N 9/22; C12N 15/11; A61P 35/00

(52) U.S. Cl. CPC A61K 41/0038; A61K 31/4995; C07K 16/2827; C07K 16/2818; A61N 5/10; C12N 9/22; C12N 15/11; A61P 35/00; C12N 2310/20

(57) ABSTRACT

This disclosure relates to compositions and methods for treating cancer by modulating the artemin pathway.

### CROSS-REFERENCE TO RELATED APPLICATIONS

This application claims priority to U.S. Provisional Application No. 63/055,284, filed Jul. 22, 2020, which is incorporated herein by reference in its entirety.

### STATEMENT REGARDING FEDERALLY SPONSORED RESEARCH

This invention was made with government support under CA195075 awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Jul. 22, 2021, having the file name “20-216-WO_Sequence-Listing_ST25.txt” and is 3 kilobytes in size.

### BACKGROUND OF DISCLOSURE

### Field of Invention

This disclosure relates to compositions and methods for treating cancer by modulating the artemin pathway.

### Technical Background

Radiation therapy is widely used in the treatment of diverse types of cancer. Recent investigations have demonstrated the importance of the immune system in mediating the anti-tumor effects of radiotherapy. For example, ionizing radiation (IR) mediates anti-tumor immunity through maturation of dendritic cells (DCs) and activation of T cells by enhancing DNA-sensing mediated type I/II IFN production. Radiotherapy is not always beneficial, however: for example, recent clinical trials have shown that 90 percent of early stage breast cancer patients over age 70 do not benefit from radiation after breast-conserving surgery.

Checkpoint inhibitor therapy, a form of immuno oncology, is another treatment paradigm for cancer that leverages the body's immune system to achieve a therapeutic effect. Checkpoint inhibitors prevent checkpoint proteins (e.g., PD-1) from binding to their partner proteins or ligands (e.g., PD-L1) and thereby reverse an “off” switch mechanism that prevents immune cells from attacking cancer cells. Investigations of immune checkpoint inhibitors, such as PD-1 inhibitors have primarily focused on enhancing T-cell function, in part, through increased type II IFN production. While a number of these inhibitors have shown great clinical promise, the percentage of patients estimated to respond to currently available checkpoint inhibitor drugs was only 12.46% in 2018.

In light of the limited efficacy of radiotherapy and checkpoint inhibitor therapy for many individuals, there is a need to improve outcomes for individuals being treated with radiotherapy and/or checkpoint inhibitor therapy. Therefore, new approaches are needed to maximize the efficacy of radiotherapy and checkpoint inhibitor therapy.

### SUMMARY OF THE DISCLOSURE

The present disclosure describes compositions and methods of treating cancer by modulating the artemin pathway.

As described below, in one aspect, the disclosure provides a method of treating cancer in a subject, comprising administering to the subject an effective amount of at least one of a radiotherapy and a checkpoint inhibitor; and administering to the subject an effective amount of an inhibitor of the artemin pathway.

In one embodiment of the first aspect, the method comprises administering a radiotherapy. In another embodiment, the method comprises administering a checkpoint inhibitor. In another embodiment, the method comprises administering a radiotherapy and a checkpoint inhibitor.

In some embodiments of the first aspect, the checkpoint inhibitor is an antibody or antigen-binding fragment thereof. In some embodiments, the checkpoint inhibitor is a peptide. In some embodiments, the checkpoint inhibitor is a small molecule. In some embodiments, the checkpoint inhibitor inhibits PD-L1. In some embodiments, the checkpoint inhibitor is an anti-PD-L1 antibody or antigen-binding fragment thereof. In some embodiments, the checkpoint inhibitor is a small molecule that inhibits PD-L1.

In some embodiments of the first aspect, the inhibitor of the artemin pathway is an antibody or an antigen-binding fragment thereof. In some embodiments, the inhibitor of the artemin pathway is a small molecule. In some embodiments, the inhibitor of the artemin pathway is a gene editing composition. In some embodiments, the gene editing composition comprises CRISPR/Cas9. In some embodiments, the gene editing composition inhibits RET or GFRα3. In some embodiments, the inhibitor of the artemin pathway inhibits artemin. In some embodiments, the artemin inhibitor is an anti-artemin antibody or antigen-binding fragment thereof. In some embodiments, the inhibitor of the artemin pathway inhibits GFRα3. In some embodiments, the inhibitor of the artemin pathway inhibits RET. In some embodiments, the RET inhibitor is one or more of vandetanib, cabozantinib, RXDX-105, lenvatinib, sorafenib, sunitinib, dovitinib, alectinib, ponatinib, regorafenib, nintedanib, apatinib, motesanib, BLU-667, or LOXO-292. In some embodiments, the RET inhibitor is LOXO-292.

In some embodiments of the first aspect, the cancer is lung cancer, colon cancer, or melanoma. In some embodiments of the first aspect, the checkpoint inhibitor and the inhibitor of the artemin pathway are in the same composition.

In some embodiments of the first aspect, the checkpoint inhibitor and/or the inhibitor of the artemin pathway are administered subsequent to the radiotherapy. In some embodiments, the checkpoint inhibitor and/or the inhibitor of the artemin pathway are administered 3-10 days subsequent to the start of the administration of the radiotherapy. In some embodiments, the inhibitor of the artemin pathway is administered simultaneously with the checkpoint inhibitor and/or the radiotherapy.

In some embodiments of the first aspect, the inhibitor of the artemin pathway is administered subsequent to the checkpoint inhibitor and/or the radiotherapy. In some embodiments, the inhibitor of the artemin pathway is administered no more than 7 days subsequent to the checkpoint inhibitor and/or the radiotherapy.

In some embodiments of the first aspect, the checkpoint inhibitor is administered to the subject at more than one time. In some embodiments, the checkpoint is administered every other week. In some embodiments, the checkpoint inhibitor is administered every other week simultaneously with the radiotherapy. In some embodiments, the checkpoint inhibitor is administered every other week subsequent to the radiotherapy.

In some embodiments of the first aspect, the inhibitor of the artemin pathway is administered to the subject at more than one time. In some embodiments, the inhibitor of the artemin pathway is administered every other day. In some embodiments, the inhibitor of the artemin pathway is administered every other day for 14 days simultaneously with and subsequent to radiotherapy. In some embodiments, the inhibitor of the artemin pathway is administered every day. In some embodiments, the inhibitor of the artemin pathway is administered every day. In some embodiments, the inhibitor of the artemin pathway is administered every day until remission is achieved.

In some embodiments of the first aspect, the checkpoint inhibitor is administered intravenously. In some embodiments of the first aspect, the inhibitor of the artemin pathway is administered intratumorally. In some embodiments of the first aspect, the method further comprises reducing the size of a tumor or inhibiting growth of a tumor in the subject.

In a second aspect, the disclosure provides a method of treating cancer in a subject, comprising administering to the subject an effective amount of ionizing radiation, an effective amount of an anti-PD-L1 antibody or an antigen-binding fragment thereof, and an effective dose of an anti-artemin antibody or an antigen-binding fragment thereof; and reducing the size of a tumor or inhibiting growth of a tumor in the subject.

In a third aspect, the disclosure provides a method of treating cancer in a subject, comprising administering to the subject an effective amount of ionizing radiation and an effective amount of LOXO-292; and reducing the size of a tumor or inhibiting growth of a tumor in the subject.

In a fourth aspect, the disclosure provides a method of treating cancer in a subject, comprising administering to the subject an effective amount of an anti-PD-L1 antibody or an antigen-binding fragment thereof and an effective amount of LOXO-292; and reducing the size of a tumor or inhibiting growth of a tumor in the subject.

In a fifth aspect, the disclosure provides a composition, comprising: an effective amount of a checkpoint inhibitor; an effective amount of an inhibitor of the artemin pathway; and a pharmaceutically acceptable carrier, solvent, adjuvant, diluent, or combination thereof.

In some embodiments of the fifth aspect, the checkpoint inhibitor is an antibody or an antigen-binding fragment thereof. In some embodiments, the checkpoint inhibitor is a peptide. In some embodiments, the checkpoint inhibitor is a small molecule.

In some embodiments of the fifth aspect, the inhibitor of the artemin pathway is an antibody or an antigen-binding fragment thereof. In some embodiments, the inhibitor of the artemin pathway is a small molecule. In some embodiments, the inhibitor of the artemin pathway is a gene editing composition.

In some embodiments of the fifth aspect, the checkpoint inhibitor is a PD-L1 inhibitor. In some embodiments of the fifth aspect, the inhibitor of the artemin pathway is LOXO-292.

In a sixth aspect, the disclosure provides a composition, comprising an effective amount of an anti-PD-L1 antibody or an antigen-binding fragment thereof; an effective amount of an anti-artemin antibody or an antigen-binding fragment thereof; and a pharmaceutically acceptable carrier, solvent, adjuvant, diluent, or combination thereof.

In a seventh aspect, the disclosure provides a composition, comprising an effective amount of an anti-PD-L1 antibody or an antigen-binding fragment; an effective amount of LOXO-292; and a pharmaceutically acceptable carrier, solvent, adjuvant, diluent, or combination thereof.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1. Size and hematoxylin and eosin (H&E) staining of spleen. B6 mice were inoculated subcutaneously (s.c.) with Lewis lung carcinoma (LLC) cells on day 0. On day 10, tumors received one dose of 20 gray (Gy) ionizing radiation (IR) and on day 20, the spleens were harvested. Representative data are shown from two or three experiments conducted with 3-5 mice per group. Scale bars represent 1 cm in upper panel and 2 mm in lower panel.

FIG. 2. Spleen weight and total number of splenocytes. B6 mice were inoculated s.c. with LLC cells on day 0. On day 10, tumors received one dose of 20 Gy ionizing radiation (IR), and on day 20, the spleens were harvested. Representative data are shown from two or three experiments conducted with 3-5 mice per group. Data are represented as mean±standard deviation (SD). *p<0.05, **p<0.01, and ***p<0.001.

FIGS. 3A-3B. (FIG. 3A) Expression of CD45, CD71, and Ter119 on tumor-induced CD45-Ter119+CD71+ erythroid progenitor cells (Ter-cells) as analyzed by flow cytometry. (FIG. 3B) Percentage and number of Ter-cells in spleen of mice inoculated with LLC. B6 mice were inoculated s.c. with LLC cells on day 0. On day 10, tumors received one dose of 20 Gy ionizing radiation (IR), and on day 20, the spleens were harvested. Representative data are shown from two or three experiments conducted with 3-5 mice per group. Data are represented as mean±SD. *p<0.05, **p<0.01, and ***p<0.001.

FIG. 4. Percentage and number of splenic CD45⁺ erythroid progenitor cells (EPCs) on day 10 post IR. B6 mice were inoculated s.c. with LLC cells on day 0. On day 10, tumors received one dose of 20 Gy ionizing radiation (IR). Representative data are shown from two or three experiments conducted with 3-5 mice or samples per group. Data are represented as mean±SD. *p<0.05, **p<0.01, and ***p<0.001.

FIG. 5. Percentage and number of splenic CD45+ immune cells on day 10 post IR. B6 mice were inoculated s.c. with LLC cells on day 0. On day 10, tumors received one dose of 20 Gy ionizing radiation (IR). Representative data are shown from two or three experiments conducted with 3-5 mice or samples per group. Data are represented as mean±SD. *p<0.05, **p<0.01, and ***p<0.001.

FIG. 6. Percentage of Ter-cells in spleen, peripheral blood (PBL), liver, lung, bone marrow (BM) and tumor tissue of tumor-bearing mice on day 10 post IR. B6 mice were inoculated s.c. with LLC cells on day 0. On day 10, tumors received one dose of 20 Gy ionizing radiation (IR). Representative data are shown from two or three experiments conducted with 3-5 mice or samples per group. Data are represented as mean±SD. *p<0.05, **p<0.01, and ***p<0.001.

FIGS. 7A-7B. (FIG. 7A) Number of Ter-cells in spleen of mice inoculated with MC38. (FIG. 7B) Number of Ter-cells in spleen of mice inoculated with B16-SIY. B6 mice were inoculated s.c. with MC38 (7A) or B16-SIY cells (7B) on day 0. On day 10, tumors received one dose of 20 Gy ionizing radiation (IR) and on day 20, the spleens were harvested. Representative data are shown from two or three experiments conducted with 3-5 mice per group. Data are represented as mean±SD. *p<0.05, **p<0.01, and ***p<0.001.

FIGS. 8A-8B. (FIG. 8A) Number of Ter-cells in spleen of mice at various times post-IR. B6 mice were inoculated s.c. with LLC cells on day 0. On day 10, tumors received one dose of 20 Gy ionizing radiation (IR) and on the indicated time, the spleens were harvested. Representative data are shown from two or three experiments conducted with 3-5 mice per group. Data are represented as mean±SD. *p<0.05, **p<0.01, and ***p<0.001. (FIG. 8B) Size of spleen of tumor-bearing mice at various times post IR. B6 mice were inoculated s.c. with LLC cells on day 0. On day 10, tumors received one dose of 20 Gy ionizing radiation (IR). Representative spleens are shown. Scale bar represents 1 cm.

FIG. 9. Percentage of Ter-cells in spleen of mice on day 20 post IR. B6 mice were inoculated s.c. with LLC cells on day 0. On day 10, tumors received one dose of 20 Gy ionizing radiation (IR). Representative data are shown from two or three experiments conducted with 3-5 mice or samples per group. Data are represented as mean±SD. *p<0.05, **p<0.01, and ***p<0.001.

FIG. 10. Effect of IR on tumor growth. B6 mice were inoculated s.c. with LLC cells on day 0. On day 10, tumors received one dose of 20 Gy ionizing radiation (IR). Representative data are shown from two or three experiments conducted with 3-5 mice or samples per group. Data are represented as mean±SD. *p<0.05, **p<0.01, and ***p<0.001.

FIG. 11. Schematic of dual tumor model.

FIG. 12. Number of Ter-cells in the spleen. MC38 tumors were inoculated as indicated, and on day 10, tumors on the right flank were irradiated with 20 Gy. On day 10 post-IR, total tumor volume (mm³) and the number of Ter-cells in the spleen were analyzed. Representative data are shown from two or three experiments conducted with 3-5 mice per group. Data are represented as mean±SD. *p<0.05, **p<0.01, and ***p<0.001.

FIGS. 13A-13B. (FIG. 13A). Artemin expression in spleen of LLC-bearing mice. Expression was analyzed by quantitative polymerase chain reaction (qPCR) on day 10 post-IR. (FIG. 13B). Artemin (ARTN) protein levels in serum of LLC-bearing mice. Protein levels were analyzed by enzyme-linked immunosorbent assay (ELISA) on day 10 post-IR. Representative data are shown from two or three experiments conducted with 3-5 mice per group. Data are represented as mean±SD. *p<0.05, **p<0.01, and ***p<0.001.

FIG. 14. Artemin protein levels in tumor tissue of LLC-bearing mice. Protein levels were analyzed by ELISA on day 10 post-IR. Representative data are shown from two or three experiments conducted with 3-5 mice per group. Data are represented as mean±SD. *p<0.05, **p<0.01, and ***p<0.001.

FIG. 15. Artemin mRNA expression in LLC tumor cells treated with irradiation. Representative data are shown from two or three experiments conducted with 3-5 mice or samples per group. Data are represented as mean±SD. *p<0.05, **p<0.01, and ***p<0.001.

FIG. 16. TGF-β1 in serum of LLC or MC38 tumor-bearing mice on day 3 post IR. Representative data are shown from two or three experiments conducted with 3-5 mice or samples per group. Data are represented as mean±SD. *p<0.05, **p<0.01, and ***p<0.001.

FIG. 17. Size of spleen derived from wild type (WT) and interferon alpha receptor knock-out mice (IFNAR KO) mice (left panel); number of splenocytes and Ter-cells (middle and right panels). The number of Ter-cells in spleen was analyzed by flow cytometry. B6 mice were inoculated with LLC cells. On day 10 post-inoculation, tumors received one dose of 20 Gy IR, and on day 20, the spleens were harvested. Representative data are shown from two or three experiments conducted with 3-5 mice per group. Data are represented as mean±SD. *p<0.05, **p<0.01, and ***p<0.001. Scale bar represents 1 cm.

FIGS. 18A-18B. (FIG. 18A) Number of Ter-cells in the spleen as analyzed by flow cytometry: IFNAR blocking antibody study. Tumor-bearing mice were treated with IR and/or IFNAR blocking antibody. B6 mice were inoculated with LLC cells. On day 10 post-inoculation, tumors received one dose of 20 Gy IR, and on day 20, the spleens were harvested. Representative data are shown from two or three experiments conducted with 3-5 mice per group. Data are represented as mean±SD. *p<0.05, **p<0.01, and ***p<0.001. (FIG. 18B) Size of spleen. LLC Tumor-bearing mice were treated with IR and/or IFNAR blocking antibody. Scale bar represents 1 cm.

FIGS. 19A-19B. (FIG. 19A) Number of Ter-cells in the spleen as analyzed by flow cytometry: interferon alpha (IFN-α) study. Tumor-bearing mice were treated with IR and/or IFN-α. B6 mice were inoculated with LLC cells. On day 10 post-inoculation, tumors received one dose of 20 Gy IR, and on day 20, the spleens were harvested. Representative data are shown from two or three experiments conducted with 3-5 mice per group. Data are represented as mean±SD. *p<0.05, **p<0.01 and ***p<0.001. (FIG. 19B) Percentage and number of Ter-cells in spleen as analyzed by flow cytometry. B16-SIY Tumor-bearing mice were treated with IR and/or IFN-α. Representative data are shown from two or three experiments conducted with 3-5 mice or samples per group. Data are represented as mean±SD. *p<0.05, **p<0.01, and ***p<0.001.

FIG. 20. Number of Ter-cells in the spleen derived from WT and Rag1 knockout (Rag KO) mice as analyzed by flow cytometry. B6 mice were inoculated with LLC cells. On day 10 post-inoculation, tumors received one dose of 20 Gy IR, and on day 20, the spleens were harvested. Representative data are shown from two or three experiments conducted with 3-5 mice per group. Data are represented as mean±SD. *p<0.05, **p<0.01, and ***p<0.001.

FIGS. 21A-21B. Number of Ter-cells in the spleen as analyzed by flow cytometry. Tumor-bearing mice were treated with IR and/or depleting antibodies against CD8 (FIG. 21A) or CD4 (FIG. 21B). B6 mice were inoculated with LLC cells. On day 10 post-inoculation, tumors received one dose of 20 Gy IR, and on day 20, the spleens were harvested. Representative data are shown from two or three experiments conducted with 3-5 mice per group. Data are represented as mean±SD. *p<0.05, **p<0.01, and ***p<0.001.

FIG. 22. IFN-γ expression in splenic CD8⁺ T cells as analyzed by intracellular staining and flow cytometry. B6 mice were inoculated with LLC cells. On day 10 post-inoculation, tumors received one dose of 20 Gy IR, and on day 20, the spleens were harvested. Representative data are shown from two or three experiments conducted with 3-5 mice per group. Data are represented as mean±SD. *p<0.05, **p<0.01, and ***p<0.001.

FIG. 23. Number of Ter-cells in the spleen derived from WT and IFN-γ KO mice as analyzed by flow cytometry. B6 mice were inoculated with LLC cells. On day 10 post-inoculation, tumors received one dose of 20 Gy IR, and on day 20, the spleens were harvested. Representative data are shown from two or three experiments conducted with 3-5 mice per group. Data are represented as mean±SD. *p<0.05, **p<0.01, and ***p<0.001.

FIGS. 24A-24B. Apoptosis of splenic Ter-cells as analyzed by flow cytometry at various times post-IR. (FIG. 24A) representative scatterplots. (FIG. 24B) % apoptotic Ter-cells. Representative data are shown from two or three experiments conducted with 3-5 mice per group. Data are represented as mean±SD. *p<0.05, **p<0.01, and ***p<0.001.

FIG. 25. Apoptosis of splenic CD45⁺ immune cells as analyzed by flow cytometry at various times post IR. Representative data are shown from two or three experiments conducted with 3-5 mice or samples per group. Data are represented as mean±SD.

FIG. 26. Apoptosis of splenic Ter-cells of WT and IFN-γ KO mice as analyzed by flow cytometry on day 10 post-IR. Representative data are shown from two or three experiments conducted with 3-5 mice per group. Data are represented as mean±SD. *p<0.05, **p<0.01, and ***p<0.001.

FIG. 27. Apoptosis of splenic Ter-cells as analyzed by flow cytometry at indicated times post-injection. B6 mice were inoculated with LLC on day 0 and recombinant mouse IFN-γ was administered through intrasplenic injection on day 15. Representative data are shown from two or three experiments conducted with 3-5 mice per group. Data are represented as mean±SD. *p<0.05, **p<0.01, and ***p<0.001.

FIG. 28A-28C. Mechanism of IR-induced Ter-cell reduction in spleen. (FIG. 28A) Apoptosis of splenic CD45⁺ immune cells, MDSCs, and CD8⁺ T cells as analyzed by flow cytometry at indicated time post IFN-γ intrasplenic injection. (FIG. 28B) The percentage of CD8⁺ T cells were analyzed by flow cytometry at indicated time post IFN-γ intrasplenic injection. (FIG. 28C) MEW I expression on splenic Ter-cells on day 3 post IFN-γ intrasplenic injection. Representative data are shown from two or three experiments conducted with 3-5 mice or samples per group. Data are represented as mean±SD. *p<0.05, **p<0.01, and ***p<0.001.

FIG. 29. Size and H&E staining of spleens. B6 mice were inoculated with LLC cells. On day 10 post-inoculation, tumor-bearing mice were treated with either one dose of 20 Gy IR or 200 μg anti-PD-L1 (10F.9G2) by intraperitoneal (i.p.) every other day for a total of four doses. On day 20 post-inoculation, spleens were harvested. Representative spleens are shown. Scale bars represent 1 cm in upper panel and 2 mm in lower panel.

FIG. 30. Spleen weight and total number of splenocytes. B6 mice were inoculated with LLC cells. On day 10 post-inoculation, tumor-bearing mice were treated with either one dose of 20 Gy IR or 200 μg anti-PD-L1 (10F.9G2) i.p. every other day for a total of four doses. On day 20 post-inoculation, spleens were harvested. Representative data are shown from two or three experiments conducted with 3-7 mice per group. Data are represented as mean±SD. *p<0.05, **p<0.01, and ***p<0.001.

FIGS. 31A-31B. (FIG. 31A) Percentage of Ter-cells in the spleen as analyzed by flow cytometry. (FIG. 31B) Number of Ter-cells in the spleen as analyzed by flow cytometry. B6 mice were inoculated with LLC cells. On day 10 post-inoculation, tumor-bearing mice were treated with either one dose of 20 Gy IR or 200 μg anti-PD-L1 (10F.9G2) i.p. every other day for a total of four doses. On day 20 post-inoculation, spleens were harvested. Representative data are shown from two or three experiments conducted with 3-7 mice per group. Data are represented as mean±SD. *p<0.05, **p<0.01, and ***p<0.001.

FIGS. 32A-32B. (FIG. 32A) Artemin levels in the serum of mice as analyzed by ELISA on day 10 post-IR or anti-PD-L1 treatment. B6 mice were inoculated with LLC cells. On day 10 post-inoculation, tumor-bearing mice were treated with either one dose of 20 Gy IR or 200 μg anti-PD-L1 (10F.9G2) i.p. every other day for a total of four doses. On day 20 post-inoculation, spleens were harvested. Representative data are shown from two or three experiments conducted with 3-7 mice per group. Data are represented as mean±SD. *p<0.05, **p<0.01 and ***p<0.001. (FIG. 32B) Splenic artemin mRNA expression in LLC tumor-bearing mice as detected by qPCR on day 10 post treatments. Representative data are shown from two or three experiments conducted with 3-5 mice or samples per group. Data are represented as mean±SD. *p<0.05, **p<0.01 and ***p<0.001.

FIG. 33. The number of Ter-cells in spleen derived from WT and IFNAR KO mice as analyzed by flow cytometry. B6 mice were inoculated with LLC cells. On day 10 post-inoculation, tumor-bearing mice were treated with either one dose of 20 Gy IR or 200 μg anti-PD-L1 (10F.9G2) i.p. every other day for a total of four doses. On day 20 post-inoculation, spleens were harvested. Representative data are shown from two or three experiments conducted with 3-7 mice per group. Data are represented as mean±SD. *p<0.05, **p<0.01, and ***p<0.001.

FIG. 34. The number of Ter-cells in spleen derived from WT and Rag KO mice as analyzed by flow cytometry. B6 mice were inoculated with LLC cells. On day 10 post-inoculation, tumor-bearing mice were treated with either one dose of 20 Gy IR or 200 μg anti-PD-L1 (10F.9G2) i.p. every other day for a total of four doses. On day 20 post-inoculation, spleens were harvested. Representative data are shown from two or three experiments conducted with 3-7 mice per group. Data are represented as mean±SD. *p<0.05, **p<0.01, and ***p<0.001.

FIGS. 35A-35B. (FIG. 35A) Spleen size of tumor-bearing WT and Rag KO mice on day 10 post treatments. (FIG. 35B) Percentage of Ter-cells in spleen of tumor-bearing WT and Rag KO mice as analyzed by flow cytometry. Representative data are shown from two or three experiments conducted with 3-5 mice or samples per group.

FIG. 36. Number of Ter-cells in the spleen as analyzed by flow cytometry. Tumor-bearing mice were treated with either IR or PD-L1 blockade, and/or the addition of CD8 depleting antibody. B6 mice were inoculated with LLC cells. On day 10 post-inoculation, tumor-bearing mice were treated with either one dose of 20 Gy IR or 200 μg anti-PD-L1 (10F.9G2) i.p. every other day for a total of four doses. On day 20 post-inoculation, spleens were harvested. Representative data are shown from two or three experiments conducted with 3-7 mice per group. Data are represented as mean±SD. *p<0.05, **p<0.01, and ***p<0.001.

FIGS. 37A-37B. (FIG. 37A) Number of Ter-cells in spleens derived from WT and IFN-γ KO mice as analyzed by flow cytometry. B6 mice were inoculated with LLC cells. On day 10 post-inoculation, tumor-bearing mice were treated with either one dose of 20 Gy IR or 200 μg anti-PD-L1 (10F.9G2) i.p. every other day for a total of four doses. On day 20 post-inoculation, spleens were harvested. Representative data are shown from two or three experiments conducted with 3-7 mice per group. Data are represented as mean±SD. *p<0.05, **p<0.01 and ***p<0.001. (FIG. 37B) Number and percentage of splenocytes and Ter-cells of tumor-bearing mice treated with IFN-γ neutralizing antibody and either IR or PD-L1 blockade as analyzed by flow cytometry. Representative data are shown from two or three experiments conducted with 3-5 mice or samples per group. Data are represented as mean±SD. *p<0.05, **p<0.01, and ***p<0.001.

FIG. 38. Number of Ter-cells in spleens derived from WT and PD-L1 KO mice as analyzed by flow cytometry. B6 mice were inoculated with LLC cells. On day 10 post-inoculation, tumor-bearing mice were treated with either one dose of 20 Gy IR or 200 μg anti-PD-L1 (10F.9G2) i.p. every other day for a total of four doses. On day 20 post-inoculation, spleens were harvested. Representative data are shown from two or three experiments conducted with 3-7 mice per group. Data are represented as mean±SD. *p<0.05, **p<0.01, and ***p<0.001.

FIGS. 39A-39B. (FIG. 39A) Percentage of splenic CD4⁺ T cells in WT or IFNAR KO mice treated with IR as analyzed by flow cytometry. (FIG. 39B) Percentage of splenic CD8⁺ T cells in WT or IFNAR KO mice treated with IR as analyzed by flow cytometry. Representative data are shown from two or three experiments conducted with 3-5 mice or samples per group. Data are represented as mean±SD. *p<0.05, **p<0.01 and ***p<0.001.

FIG. 40. Colony formation assay of MC38 cells treated with 0, 1, or 3 Gy irradiation. MC38 cells were either co-cultured with 2×10⁶ Ter-cells (left) sorted from spleens of tumor-bearing mice, or treated with 100 ng/mL artemin (right). Representative data are shown from three experiments. Data are represented as mean±SD. *p<0.05, **p<0.01, and ***p<0.001.

FIGS. 41A-41B. (FIG. 41A) Apoptosis of MC38 cells as analyzed by flow cytometry. 1×10⁵ MC38-OTI-zsGreen cells were co-cultured with 2×10⁵ CD8⁺ T cells purified from OTI mice in 96-well U bottomed plates. Tumor cells were either co-cultured with 2×10⁵ Ter-cells sorted from spleens of tumor-bearing mice or treated with 100 ng/mL artemin for 6 h. Representative data are shown from three experiments. Data are represented as mean±SD. *p<0.05, **p<0.01 and ***p<0.001. (FIG. 41B) Apoptosis of MC38 cells was analyzed by flow cytometry. 1×10⁵ MC38-OTI-zsGreen cells were co-cultured with 2×10⁵ CD8+ T cells purified from OTI mice in 96-well U bottom plates. Tumor cells were either co-cultured with 2×10⁵ Ter-cells sorted from spleen of tumor-bearing mice or treated with 100 ng/mL artemin (ARTN) for 6 h. Representative data are shown from two or three experiments conducted with 3-5 mice or samples per group.

FIG. 42. Tumor growth monitoring: Ter-cell treatment. B6 mice were inoculated with LLC cells. On day 10 post-inoculation, tumor-bearing mice were treated with IR or anti-PD-L1. Mice were transferred i.v. with 1×10⁷ purified Ter-cells every other day for a total of three times. Representative data are shown from three experiments. Data are represented as mean±SD. *p<0.05, **p<0.01, and ***p<0.001.

FIG. 43. Tumor growth monitoring: artemin treatment. B6 mice were inoculated with LLC cells. On day 10 post-inoculation, tumor-bearing mice were treated with IR or anti-PD-L1. Mice were treated intra-tumor injection (i.t.) with 0.5 μg/mouse artemin (ARTN) every other day. Representative data are shown from three experiments. Data are represented as mean±SD. *p<0.05, **p<0.01, and ***p<0.001.

FIG. 44. Tumor growth of LLC tumor-bearing WT, IFNAR KO (left), and IFN-γ KO (right) mice treated with IR on day 10 post tumor inoculation. Representative data are shown from two or three experiments conducted with 3-5 mice or samples per group. Data are represented as mean±SD. *p<0.05, **p<0.01, and ***p<0.001.

FIG. 45. Percentage and number of Ter-cells in spleen were analyzed by flow cytometry. B6 mice were treated i.v. or s.c. with 20 U/mouse recombinant erythropoietin (EPO) every other day for a total of 6 days. Representative spleen size (left) and data (right) are shown from two or three experiments conducted with 3-5 mice or samples per group. Data are represented as mean±SD. *p<0.05, **p<0.01, and ***p<0.001. Scale bar represents 1 cm.

FIGS. 46A-46B. Number of Ter-cells in spleen as analyzed by flow cytometry: IR and anti-PD-L1 treatments. B6 mice were inoculated with LLC cells. On day 10 post-inoculation, tumor-bearing mice were treated with IR (FIG. 46A), or anti-PD-L1 (FIG. 46B). Mice were treated i.v. with 20 U/mouse EPO every other day. Representative data are shown from three experiments. Data are represented as mean±SD. *p<0.05, **p<0.01, and ***p<0.001.

FIG. 47. Artemin levels in serum as determined by ELISA on day 10 post-treatments. B6 mice were inoculated with LLC cells. On day 10 post-inoculation, tumor-bearing mice were treated with IR or anti-PD-L1. Mice were treated i.v. with 20 U/mouse EPO every other day. Representative data are shown from three experiments. Data are represented as mean±SD. *p<0.05, **p<0.01, and ***p<0.001.

FIGS. 48A-48B. Tumor growth of LLC tumor-bearing mice treated with EPO and either IR (FIG. 48A) or anti-PD-L1 (FIG. 48B). Representative data are shown from three experiments. Data are represented as mean±SD. *p<0.05, **p<0.01, and ***p<0.001. N. S, not significant.

FIG. 49. Percentage and number of Ter-cells in spleen as analyzed by flow cytometry. LLC tumor-bearing mice were treated i.p. with 20 μg/mouse anti-Ter119 every other day for a total of 6 days. Representative data are shown from two or three experiments conducted with 3-5 mice or samples per group. Data are represented as mean±SD. *p<0.05, **p<0.01, and ***p<0.001.

FIGS. 50A-50B. (FIG. 50A) Tumor growth monitoring: IR, EPO, and/or anti-Ter119 treatment. B6 mice were inoculated with LLC cells and treated with IR, EPO, and/or anti-Ter119. (FIG. 50B) Tumor growth monitoring: anti-PD-L1, EPO, and/or anti-Ter119 treatment. B6 mice were inoculated with LLC cells and treated with anti-PD-L1, EPO, and/or anti-Ter119. Representative data are shown from three experiments. Data are represented as mean±SD. *p<0.05, **p<0.01, and ***p<0.001.

FIG. 51. Artemin (ARTN) levels in serum as determined by ELISA on day 10 post-treatments. B6 mice were inoculated with LLC cells. On day 10 post-inoculation, tumor-bearing mice were treated with either IR or anti-PD-L1. Splenectomy was performed 1 day before treatments. Representative data are shown from two or three experiments. Data are represented as mean±SD. *p<0.05, **p<0.01, and ***p<0.001.

FIG. 52. Tumor growth of LLC tumor-bearing mice treated with splenectomy and IR. Representative data are shown from two or three experiments. Data are represented as mean±SD. *p<0.05, **p<0.01, and ***p<0.001.

FIG. 53. Tumor growth of LLC tumor-bearing mice treated with splenectomy and anti-PD-L1. Representative data are shown from two or three experiments. Data are represented as mean±SD. *p<0.05, **p<0.01, and ***p<0.001.

FIG. 54. Number of lung nodules in LLC tumor-bearing mice at the end of tumor growth measurement: IR treatment. B6 mice were inoculated with LLC cells. On day 10 post inoculation, tumor-bearing mice were treated with IR. Splenectomy was performed 1 day before treatments. Representative data are shown from two or three experiments conducted with 3-5 mice or samples per group. Data are represented as mean±SD. **p<0.01 and ***p<0.001.

FIG. 55. Number of lung nodules in LLC tumor-bearing mice at the end of tumor growth measurement: anti-PD-L1 treatment. B6 mice were inoculated with LLC cells. On day 10 post inoculation, tumor-bearing mice were treated with anti-PD-L1. Splenectomy was performed 1 day before treatments. Representative data are shown from two or three experiments conducted with 3-5 mice or samples per group. Data are represented as mean±SD. **p<0.01 and ***p<0.001.

FIG. 56. Tumor growth of LLC tumor-bearing mice treated with Ter119-depleting antibody and/or IR. Representative data are shown from two or three experiments. Data are represented as mean±SD. *p<0.05, **p<0.01, and ***p<0.001.

FIG. 57. Tumor growth of LLC tumor-bearing mice treated with Ter119-depleting antibody and/or anti-PD-L1. Representative data are shown from two or three experiments. Data are represented as mean±SD. *p<0.05, **p<0.01, and ***p<0.001.

FIG. 58A-58C. (FIG. 58A) Tumor growth of LLC tumor-bearing mice treated with artemin-neutralizing antibody i.t. and either IR or anti-PD-L1. (FIG. 58B) Western blot for GFRα3. shRNA were used to generate GFRα3 Knockdown (KD) MC38 cell lines. The expression of GFRα3 in MC38 cells were analyzed with western blot. Clone 8865 was used for following tumor model. (FIG. 58C) Tumor growth in GFRα3 KD MC38 cells with treatment. WT or GFRα3 KD MC38 cells were inoculated in B6 mice, and tumor growth was monitored following treatment with either IR or anti-PD-L1.

Representative data are shown from two or three experiments. Data are represented as mean±SD. *p<0.05, **p<0.01, and ***p<0.001.

FIG. 59. Expression of RET in MC38 cells as analyzed with western blot. CRISPR/Cas9 was used to generate RET KO MC38 cell lines. Representative data are shown from two or three experiments conducted with 3-5 mice or samples per group. Data are represented as mean±SD. **p<0.01 and ***p<0.001.

FIG. 60. Tumor growth monitoring: IR treatment. WT or RET KO MC38 cells were inoculated in B6 mice, and tumor growth was monitored following treatment with either IR or anti-PD-L1. Representative data are shown from two or three experiments. Data are represented as mean±SD. *p<0.05, **p<0.01, and ***p<0.001.

FIGS. 61A-61C. Tumor growth of LLC tumor-bearing mice treated with LOXO-292 and either IR (FIG. 61A) or anti-PD-L1 (FIG. 61B). (FIG. 61C) p-AKT and p-ERK in LLC cells treated with 100 ng/mL ARTN and 1 μg/mL LOXO-292 were detected by western blot. Representative data are shown from two or three experiments. Data are represented as mean±SD. *p<0.05, **p<0.01, and ***p<0.001.

FIGS. 62A-62B. Number of lung modules. B6 mice were inoculated with LLC cells. On day 10 post inoculation, tumor-bearing mice were treated with either IR (FIG. 62A) or anti-PD-L1 (FIG. 62B) plus LOXO-292. Splenectomy was performed 1 day before treatments. The number of lung nodules were observed in LLC tumor-bearing mice at the end of tumor growth measurement. Representative data are shown from two or three experiments conducted with 3-5 mice or samples per group. Data are represented as mean±SD. *p<0.05, **p<0.01, and ***p<0.001.

FIGS. 63A-63B. Tumor growth monitoring. (FIG. 63A) IR treatment. B6 mice were inoculated with LLC cells and treated with IR, IR and EPO or IR, EPO, and LOXO-292. (FIG. 63B) anti-PD-L1 treatment. B6 mice were inoculated with LLC cells and treated with either anti-PD-L1, anti-PD-L1 and EPO, or anti-PD-L1, EPO, and LOXO-292. Representative data are shown from two or three experiments. Data are represented as mean±SD. *p<0.05, **p<0.01, and ***p<0.001.

FIG. 64. Artemin (ARTN) levels in the serum of lung cancer patients prior to (pre-RT) and immediately following (post-RT) definitive chemoradiation therapy (“RT”) as determined by ELISA. Response was defined by imaging at time of follow up with “non-progressors” having no evidence of disease at most recent follow up examination and “progressors” having progression of disease on post-treatment imaging. Each line denotes an individual patient.

FIG. 65. Pre-treatment tumor GFRα3 expression in two cohorts of patients with metastatic melanoma treated with immune checkpoint blockade (studies of the cohorts are published under Riaz et al., Cell 2017 Nov. 2; 171(4):934-949, PubMed ID 29033130; and Gide et al., Cancer Cell 2019 Feb. 11; 35(2):238-255, PubMed ID 30753825). Median pre-treatment GFRα3 expression was used to split patients into low and high expressing groups. CR/PR, complete response/partial response denotes >30% shrinkage; PD, progressive disease denotes >20% growth. P-value determined using Pearson Chi-Square test. Compared immunotherapy response rates by GFRα3 expression are shown.

FIG. 66. Circulating CD45−CD71+CD235a+ Ter-cell abundance in the peripheral blood of cancer patients prior to (pre-RT) and following (post-RT) radiotherapy treatment as determined by flow cytometry. Patients were treated with ablative radiotherapy followed by pembrolizumab immunotherapy in the NCT02608385 clinical trial. Response was measured using RECISTv1.1 criteria. Responders exhibited partial or complete responses, whereas non-responders exhibited disease progression following treatment. P-values determined using two-tailed paired Student's t-test. Each line denotes an individual patient.

FIGS. 67A-67B. (FIG. 67A) Relationship between change in tumor size and change in tumor GFRα3 expression in the response to radiotherapy for patients treated in the NCT02608385 clinical trial. Data represent mean+/−SEM. P-value determined using two-tailed unpaired Student's t-test. (FIG. 67B) Change in tumor GFRα3 expression as a function of clinical response to radiotherapy and pembrolizumab based on RECISTv1.1 criteria (NCT02608385 trial). CR, complete response. PR, partial response (>30% shrinkage). SD, stable disease. PD, progressive disease (>20% growth).

FIG. 68. Change in intratumoral perforin (PRF1) expression as a function of change in tumor GFRα3 expression following radiotherapy (NCT02608385 trial).

FIG. 69. Expression of GFRα3 on CD45 tumor cells as detected by flow cytometry. B6 mice were inoculated s.c. with LLC cells on day 0. On day 10, tumor-bearing mice were treated with IR or PD-L1 blockade. Tumors were harvested and expression was detected. Data are represented as mean±SD. ***p<0.001.

FIG. 70. Schema of the interaction between IR and PD-L1 blockade and tumor-induced Ter-cells. Tumor-induced Ter-cells accumulate in spleen through TGF-β signaling and, in turn, Ter-cells promote tumor progression by secreting artemin. IR decreases Ter-cell numbers in spleen through the Type I IFN—CD8+ T cells—IFN-γ axis. Similarly, PD-L1 blockade reduces Ter-cells through CD8+ T cells and IFN-γ. In contrast, Ter-cells and artemin, which can be restored by EPO during therapies, impair the efficacy of both IR and PD-L1 blockade. Furthermore, Ter-cell depletion, artemin neutralization, and artemin receptor inhibition facilitate the efficacy of IR and PD-L1 blockade.

FIGS. 71A-71I. Analysis of proliferation and effector molecules in response to artemin treatment in CD8 T cells and MC38 murine colon cancer tumors revealed that artemin directly attenuates CD8 T cell effector function in vitro and in vivo. (FIGS. 71A-71E) CD8 T cells were purified from WT spleen and treated with recombinant artemin for 24 hours (150 ng/mL for FIGS. 71B, 71C, 71D, and 71E). T proliferation was examined (FIG. 71A), and the indicated effector molecules were analyzed by flow cytometry (FIGS. 71B, 71C, and 71D) and q-PCR (FIG. 71E). (FIGS. 71F-71I) MC38 murine colon cancer tumors were treated with artemin (i.t.), and single cell suspensions of tumors were stained with indicated markers and examined by flow cytometry (FIGS. 71F, 71G, 71H, and 71I).

FIGS. 72A-72C. Analysis of artemin effects on exhaustion markers, CD4 T cells, and PD-L1 expression revealed that artemin upregulates PD-L1 expression in DCs MDSCs in established MC38 tumors. MC38 tumor were digested into single cell suspension and stained with markers that characterize T cells, DCs, and MDSCs. Flow cytometry was performed. (FIG. 72A) Artemin treatment did not affect exhaustion status of intratumoral T cells. (FIG. 72B) Changes in CD4 T cells. (FIG. 72C) Artemin induced PD-L1 expression on DCs and MDSCs in MC38 tumors.

FIGS. 73A-73D. Study of the effects of artemin and radiation on mRNA expression of possible artemin receptors in different immune cells. (FIG. 73A) mRNA level of known GDNF receptors in CD8+ T cells after co-culture with artemin. (FIG. 73B) mRNA level of known GDNF receptors in MDSCs (CD11b+Ly6C+) cells. GFRα1, GFRα3 and NCAM expression in DC (FIG. 73C) and natural killer (NK) cells (FIG. 73D) in tumors responding to artemin or/and radiation treatments. R1, GFRα1; R3, GFRα3.

### DETAILED DESCRIPTION OF PREFERRED EMBODIMENTS

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

All publications, patents, and patent applications cited herein are hereby expressly incorporated by reference in their entirety for all purposes.

### Definitions

Before describing the methods and compositions of the disclosure in detail, a number of terms will be defined. As used herein, the singular forms “a,” “an,” and “the” include plural referents unless the context clearly dictates otherwise. For example, reference to “a therapeutic target” means one or more therapeutic targets.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” can be replaced with either of the other two terms, while retaining their ordinary meanings.

Percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, typically suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

As used herein, the terms “therapeutic amount,” “therapeutically effective amount,” or “effective amount” can be used interchangeably and refer to an amount of a compound that becomes available through the appropriate route of administration to treat a patient for a disorder, a condition, or a disease. The amount of a compound which constitutes a “therapeutic amount,” “therapeutically effective amount,” or “effective amount” will vary depending on the compound, the disorder and its severity, and the age of the subject to be treated, but can be determined routinely by one of ordinary skill in the art.

“Treating” or “treatment” as used herein covers the treatment of a disease or disorder described herein, in a subject, preferably a human, and includes:
i. inhibiting a disease or disorder, i.e., arresting its progression;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder. For example, the terms “treating,” “treat,” or “treatment” refer to either preventing, providing symptomatic relief, or curing a patient's disorder, condition, or disease.

As used herein, the terms “patient” and/or “subject” and/or “individual” can be used interchangeably and refer to an animal. For example, the patient, subject, or individual can be a mammal, such as a human to be treated for a disorder, condition, or a disease.

As used herein, the terms “disorder,” “condition,” or “disease” refer to cancers, and in some embodiments, associated comorbidities.

It is noted that terms like “preferably,” “commonly,” and “typically” are not utilized herein to limit the scope of the methods and compositions as described herein or to imply that certain features are critical, essential, or even important to the structure or function of the claimed invention.

As used herein, the term “cancer” refers to any type of cancerous cell or tissue as well as any stage of a cancer from precancerous cells or tissues to metastatic cancers. For example, as used herein, cancer can refer to a solid cancerous tumor, leukemia, and/or a neoplasm.

As used herein, the term “radiotherapy” refers to administration of at least one “radiotherapeutic agent” to a subject having a tumor or cancer and refers to any manner of treatment of a tumor or cancer with a radiotherapeutic agent. A radiotherapeutic agent includes, for example, ionizing radiation including, for example, external beam radiotherapy, stereotactic radiotherapy, virtual simulation, 3-dimensional conformal radiotherapy, intensity-modulated radiotherapy, ionizing particle therapy, and radioisotope therapy.

As used herein, the term “inhibit” means to slow down or reduce the activity of a protein, enzyme, or other agent. “Inhibit” can include complete elimination of a protein or its activity. The term “inhibit” can further mean to prevent functional interaction of one or more compounds, molecules, or proteins. For example, an inhibitor can prevent a receptor from accepting its ligand or prevent activation of the receptor when accepting its ligand.

### Overview

The present inventors have unexpectedly discovered that combining radiotherapy or checkpoint inhibitor immunotherapy with artemin pathway blockade significantly enhances the efficacy of both radio- and immunotherapies compared to monotherapy or a combination of radiotherapy and immunotherapy alone.

Provided herein are methods for treating cancer using a combination of inhibition of the artemin pathway with one or both of radiotherapy and checkpoint inhibition. Embodiments of the present disclosure include methods of treating cancer in a subject comprising administering to the subject an effective dose of at least two of the following: a radiotherapy, a checkpoint inhibitor, and an inhibitor of the artemin pathway. In some embodiments, the method comprises administering a radiotherapy and an inhibitor of the artemin pathway. In some embodiments, the method comprises administering a checkpoint inhibitor and an inhibitor of the artemin pathway. In some embodiments, the method comprises administering a radiotherapy, a checkpoint inhibitor, and an inhibitor of the artemin pathway.

In some embodiments of the present disclosure, a method of treating cancer in a subject includes administering to the subject an effective amount of ionizing radiation, an effective amount of an anti-PD-L1 antibody or an antigen-binding fragment thereof, and an effective dose of an anti-artemin antibody or an antigen-binding fragment thereof, and reducing the size of a tumor or inhibiting growth of the tumor in the subject. In some embodiments of the present disclosure, a method of treating cancer in a subject includes administering to the subject an effective amount of ionizing radiation and an effective amount of LOXO-292, and reducing the size of a tumor or inhibiting growth of the tumor in the subject. Additionally, in some embodiments of the present disclosure a method of treating cancer in a subject includes administering to the subject an effective amount of an anti-PD-L1 antibody or an antigen-binding fragment thereof and an effective amount of LOXO-292, and reducing the size of a tumor or inhibiting growth of the tumor in the subject.

Also provided herein are compositions for treating cancer. In some embodiments of the present disclosure, a composition can include an effective amount of a checkpoint inhibitor, an effective amount of an inhibitor of the artemin pathway, and a pharmaceutically acceptable carrier, solvent, adjuvant, diluent, or combination thereof.

In some embodiments of the present disclosure, a composition can include an effective amount of an anti-PD-L1 antibody or an antigen-binding fragment thereof, an effective amount of an anti-artemin antibody or an antigen-binding fragment thereof, and a pharmaceutically acceptable carrier, solvent, adjuvant, diluent, or combination thereof. In some embodiments of the present disclosure a composition can include an effective amount of an anti-PD-L1 antibody or an antigen-binding fragment, an effective amount of a RET inhibitor, and a pharmaceutically acceptable carrier, solvent, adjuvant, diluent, or combination thereof. In some embodiments of the present disclosure, a composition can include an effective amount of an anti-PD-L1 antibody or an antigen-binding fragment, an effective amount of LOXO-292, and a pharmaceutically acceptable carrier, solvent, adjuvant, diluent, or combination thereof. Radiotherapy

Radiotherapy is based on ionizing radiation delivered to a target area that results in death of tumor cells. The present disclosure contemplates a variety of radiotherapy approaches. Radiotherapy that can be used herein can include the application of radiation from sources including cesium, palladium, iridium, iodine, and/or cobalt. Radiation is usually delivered as ionizing radiation delivered from a linear accelerator or an isotopic source, such as a cobalt. Specific linear accelerators (LINACs) contemplated for use herein include Cyberknife® and TomoTherapy®. Particle radiotherapy from cyclotrons, such as delivery of protons or carbon nuclei, can also be employed. In addition, radioisotopes such as ³²P or radium-223 can be delivered systemically. External radiotherapy can be systemic radiation in the form of stereotactic radiotherapy, total nodal radiotherapy, or whole body radiotherapy. However, radiation can also be focused to a particular site, such as the location of the tumor or the solid cancer tissues (for example, abdomen, lung, liver, lymph nodes, head, etc.).

The radiation dosage regimen is generally defined in terms of gray (Gy) or sieverts (Sv), time, and fractionation, and can be readily defined by a skilled radiation oncologist. The amount of radiation a subject receives will depend on various considerations, but two important considerations are 1) the location of the tumor in relation to other critical structures or organs of the body, and 2) the extent to which the tumor has spread. One illustrative example of a course of treatment for a subject undergoing radiation therapy includes a treatment schedule taking place over a 5 to 8 week period, with a total dose of 50 to 80 Gy administered to the subject in a single daily fraction of 1.8 to 2.0 Gy, 5 days a week. One Gy refers to 100 rad of dose.

Radiotherapy can also include implanting radioactive seeds inside or next to a site designated for radiotherapy and is termed brachytherapy (or internal radiotherapy, endocurietherapy, or sealed source therapy). For prostate cancer, there are currently two types of brachytherapy: permanent and temporary. In permanent brachytherapy, radioactive (iodine-125 or palladium-103) seeds can be implanted into the prostate gland using ultrasound for guidance. Illustratively, about 40 to 100 seeds are implanted, and the number and placement are generally determined by a computer-generated treatment plan known in the art specific for each subject. Temporary brachytherapy uses a hollow source placed into the prostate gland that is filled with radioactive material (iridium-192) for about 5 to about 15 minutes, for example. Following treatment, the needle and radioactive material are removed. This procedure is repeated two to three times over a course of several days.

Radiotherapy can also include radiation delivered by external beam radiation therapy (EBRT), including, for example, a linear accelerator (a type of high-powered X-ray machine that produces very powerful photons that penetrate deep into the body); proton beam therapy where photons are derived from a radioactive source such as iridium-192, caesium-137, radium-226 (no longer used clinically), or colbalt-60; Hadron therapy; multi-leaf collimator (MLC); and intensity modulated radiation therapy (IMRT). During EBRT, a brief exposure to the radiation is given for a duration of several minutes, and treatment is typically given once per day, 5 days per week, for about 5 to 8 weeks. No radiation remains in the subject after treatment. There are several ways to deliver EBRT, including, for example, three-dimensional conformal radiation therapy where the beam intensity of each beam is determined by the shape of the tumor. Illustrative dosages used for photon-based radiation are measured in Gy, and in an otherwise healthy subject (that is, little or no other disease states present such as high blood pressure, infection, diabetes, etc.) for a solid epithelial tumor ranges from about 60 to about 80 Gy, and for a lymphoma ranges from about 20 to about 40 Gy. Illustrative preventative (adjuvant) doses are typically given at about 45 to about 60 Gy in about 1.8 to about 2 Gy fractions for breast, head, and neck cancers.

When radiation therapy is a local modality, radiation therapy as a single line of therapy is unlikely to provide a cure for those tumors that have metastasized distantly outside the zone of treatment. Thus, the use of radiation therapy with other modality regimens, including chemotherapy, can have important beneficial effects for the treatment of metastasized cancers.

Radiation therapy has also been combined temporally with chemotherapy to improve the outcome of treatment. There are various terms to describe the temporal relationship of administering radiation therapy and chemotherapy, and the following examples are non-limiting illustrative treatment regimens generally known by those skilled in the art. “Sequential” radiation therapy and chemotherapy refers to the administration of chemotherapy and radiation therapy separately in time. “Simultaneous” radiation therapy and chemotherapy refers to the administration of chemotherapy and radiation therapy at the same time or more typically on the same day. “Simultaneous” administration can also refer to multiple treatments that overlap in time even if they are not co-administered at the same time or even consistently on the same day—for example, if a first treatment is given every other day and a second treatment is administered on the “off” days for the first treatment over a period, or if a first treatment is given every four days and a second treatment every three days over a period. “Alternating” radiation therapy and chemotherapy refers to the administration of radiation therapy on the days in which chemotherapy would not have been administered if it were given alone.

It should be noted that therapeutically effective doses of radiotherapy can be determined by a radiation oncologist skilled in the art and can be based on, for example, whether the subject is receiving chemotherapy, if the radiation is given before or after surgery, the type and/or stage of cancer, the type of radiotherapy to be used, the location of the tumor, and the age, weight and general health of the subject.

### Checkpoint Inhibitors

Checkpoint inhibitors work by blocking immune checkpoints that shut down immune responses and protect themselves. These molecules are able to unleash new immune responses against cancer as well as enhance existing responses to promote elimination of cancer cells. In some embodiments of the present disclosure, checkpoint inhibitors can be administered to a subject to reduce tumor-induced Ter-cell accumulation in a CD8+ T cell and IFN-γ-dependent manner. Checkpoint inhibitors are perhaps the most well-known, and most widely successful, immunomodulators developed so far. Several therapies available or in development target Cytotoxic T-Lymphocyte-Associated protein 4 (CTLA-4, also called CD152) or the Programmed Death 1 (PD-1) pathway. There are a variety of other checkpoint targets, including the following: Adenosine A2A receptor (A2AR), B7-H3 or CD276, B7-H4 or VTCN1, B and T Lymphocyte Attenuator (BTLA) or CD272, Herpesvirus Entry Mediator (HVEM), Indoleamine 2,3-dioxygenase (IDO), tryptophan 2,3-dioxygenase (TDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), V-domain Ig suppressor of T cell activation (VISTA), Sialic acid-binding immunoglobulin-type lectin 7 (SIGLEC7) or CD328, and Sialic acid-binding immunoglobulin-type lectin 9 (SIGLEC9) or CD329. The present disclosure contemplates inhibitors targeting all of these checkpoints and administration of one or more checkpoint inhibitors to a subject in need thereof. In some embodiments of the present disclosure, the checkpoint inhibitor is an antibody or antigen-binding fragment thereof. In some embodiments, the checkpoint inhibitor is a peptide. In other embodiments, the checkpoint inhibitor is a small molecule.

The present disclosure contemplates compositions and methods targeting Programmed Death-Ligand 1 (PD-L1). Antibodies, peptides, or small molecules serving as inhibitors of PD-L1 can include Atezolizumab (Tecentriq), Avelumab (Bavencio), Durvalumab (Imfinzi), KN035, CK-301, AUNP12, CA-170, BMS-986189, and other compositions. In some embodiments of the present disclosure, the checkpoint inhibitor inhibits PD-L1. In some embodiments, the PD-L1 inhibitor is an antibody or antigen-binding fragment thereof. In some embodiments, the PD-L1 inhibitor is a peptide. In some embodiments, the PD-L1 inhibitor is a small molecule.

### Inhibitors of the Artemin Pathway

The present disclosure further contemplates compositions and methods that involve inhibition of artemin and other molecules in the artemin pathway. Inhibitors of the artemin pathway can be reversible or irreversible. They can be proteins, including antibodies, or nucleic acids, or small molecules. The inhibitors of the artemin pathway contemplated by the present disclosure can target artemin itself, its receptors, or its co-receptors. For example, the inhibitors can target RET or GFR-alpha 3 (or “GFRα3”). The present disclosure contemplates the administration of LOXO-292, which is a small molecule inhibitor of RET, to a subject in need thereof. The inhibitors of the artemin pathway can also be compositions targeting cells that secrete artemin—for example, antibodies that target tumor-induced CD45-Ter119+CD71+ erythroid progenitor cells (EPCs), also known as Ter-cells. The inhibitors of the artemin pathway can also be gene editing compositions. For example, the inhibitors can be a CRISPR/Cas9 composition, or series of compositions, that knocks out RET and/or GFRα3. This disclosure contemplates use of inhibitors of the artemin pathway that perform inhibition of the pathway ex vivo or in vivo. In vivo mechanisms of achieving a CRISPR/Cas9 knock out of RET and/or GFRα3 can include viral and/or non-viral delivery mechanisms. Examples of viral delivery mechanisms include adeno-associated viral vectors (AAV) and lentiviral vectors. Examples of non-viral delivery mechanisms include cell-penetrating peptides (CPPs), lipid nanoparticles (LNPs), polymer-based particles, and inorganic encapsulating materials, such as zeolitic imidazole frameworks (ZIFs) or colloidal gold nanoparticles. In some embodiments, the CRISPR/CAS9 components are targeted to tumor cells. Targeting can be achieved by intratumoral delivery and/or molecular targeting. In some embodiments, the knockout of RET and/or GFRα3 may be partial/incomplete (i.e., not occurring in all cells in situ); radiosensitization and better response to checkpoint inhibitors are still anticipated in attenuating the pathway.

In some embodiments of the present disclosure, the inhibitor of the artemin pathway is an antibody or antigen-binding fragment thereof that specifically binds one or more molecules in the artemin pathway to interrupt its function. In other embodiments, the inhibitor of the artemin pathway is a small molecule. In other embodiments, the inhibitor of the artemin pathway is a gene editing composition. In some embodiments, the gene editing composition comprises CRISPR/Cas9. In some embodiments, the gene editing composition inhibits RET. In some embodiments, the inhibitor of the artemin pathway inhibits artemin. In some embodiments, the artemin inhibitor is an anti-artemin antibody or an antigen-binding fragment thereof. In some embodiments, the inhibitor of the artemin pathway inhibits GFRα3. In some embodiments, the inhibitor of the artemin pathway inhibits RET. In some embodiments, the inhibitor of RET is a multikinase inhibitor (MKI). In some embodiments, the inhibitor of RET is specific to mutant RET. In some embodiments, the inhibitor of RET targets oncogenic RET. In other embodiments, the inhibitor of RET targets wild-type RET. In some embodiments, the RET inhibitor is vandetanib, cabozantinib, RXDX-105, lenvatinib, sorafenib, sunitinib, dovitinib, alectinib, ponatinib, regorafenib, nintedanib, apatinib, motesanib, BLU-667, or LOXO-292. In some embodiments, the RET inhibitor is LOXO-292. Some embodiments of the present disclosure comprise more than one inhibitor of the artemin pathway. For example, some embodiments include both a small molecule inhibitor of the pathway and an anti-artemin antibody. The cancer treated by the embodiments in the present disclosure can be any cancer. In some embodiments, the cancer is melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplastic syndrome, multiple myeloma, transitional cell carcinoma, neuroblastoma, plasma cell neoplasms, Wilm's tumor, glioblastoma, retinoblastoma, or hepatocellular carcinoma. The cancer can be refractive to other treatments, such as chemotherapy, radiotherapy, and/or checkpoint inhibitors.

### Therapeutic Compositions

The therapeutic compositions of the present disclosure can take a form suitable for virtually any mode of administration, including, for example, injection, transdermal, oral, topical, ocular, buccal, systemic, nasal, rectal, vaginal, etc., or a form suitable for administration by inhalation or insufflation. Compositions that can be delivered intravenously and/or intratumorally are also contemplated herein. In some embodiments of the present disclosure, a checkpoint inhibitor is administered intravenously. In some embodiments, the inhibitor of the artemin pathway is administered intratumorally.

Compositions containing active pharmaceutical ingredients may also contain one or more inactive pharmaceutical excipients and other substances. The therapeutic compositions described herein can include a pharmaceutically acceptable carrier, solvent, adjuvant, diluent, or combination thereof. These ingredients can include, but are not limited to, lubricants, solubilizers, alcohols, binders, controlled release polymers, enteric polymers, disintegrants, colorants, flavorants, sweeteners, antioxidants, preservatives, pigments, additives, fillers, suspension agents, surfactants (for example, anionic, cationic, amphoteric and nonionic), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

The therapeutic compositions described herein, or pharmaceutical compositions thereof, will generally be used in an amount effective to achieve the intended result (“effective dose” or “effective amount”), for example, in an amount effective to treat or prevent the particular disease being treated (e.g., a therapeutically effective amount) and thereby provide a therapeutic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally can include halting or slowing the progression of the disease.

The amount of therapeutic composition administered can be based upon a variety of factors, including, for example, the particular condition being treated, the mode of administration, whether the desired benefit is prophylactic and/or therapeutic, the severity of the condition being treated and the age and weight of the patient, the genetic profile of the patient, and/or the bioavailability of the particular therapeutic composition, etc.

Determination of an effective dosage of compound(s) for a particular use and mode of administration is well within the capabilities of those skilled in the art. Effective dosages can be estimated initially from in vitro activity and metabolism assays. For example, an initial dosage of a therapeutic composition for use in animals can be formulated to achieve a circulating blood or serum concentration of the therapeutic composition that is at or above an EC₅₀ of the particular therapeutic composition as measured in an in vitro assay. Calculating dosages to achieve such circulating blood or serum concentrations taking into account the bioavailability of the particular therapeutic composition via the desired route of administration is well within the capabilities of skilled artisans. Initial dosages of therapeutic compositions can also be estimated from in vivo data, such as animal models. Animal models useful for testing the efficacy of the therapeutic composition to treat or prevent the various diseases described above are well known in the art. Animal models suitable for testing the bioavailability of the therapeutic composition are also well known. Skilled artisans can routinely adapt such information to determine dosages of particular therapeutic compositions suitable for human administration.

Dosage amounts can be in the range of from about 0.0001 mg/kg/day, 0.001 mg/kg/day, or 0.01 mg/kg/day to about 100 mg/kg/day, but can be higher or lower, depending upon, among other factors, the activity of the therapeutic agent, the bioavailability of the therapeutic composition, other pharmacokinetic properties, the mode of administration and various other factors, including particular diseases being treated, the site of the disease within the body, the severity of the disease, the genetic profile, age, health, sex, diet, and/or weight of the subject. Dosage amount and interval can be adjusted individually to provide levels of the therapeutic compositions sufficient to maintain therapeutic and/or prophylactic effects. For example, the therapeutic compositions can be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of therapeutic compositions may not be related to plasma concentration. Skilled artisans will be able to optimize effective dosages without undue experimentation.

The present disclosure contemplates different modes of administration, dosage amounts, intervals, and treatment durations. These variables can be interdependent, and the treatment regimen will depend on the judgment of the prescribing physician. In some embodiments, the mode of administration for one or more of the compositions is intratumoral injection (“intratumoral”). In some embodiments, the mode of administration for one or more of the compositions is oral. In some embodiments, the mode of administration for one or more of the compositions is intravenous. In some embodiments, the interval of administration (“interval”) for one or more of the compositions is every other day. In some embodiments, the interval of administration for one or more of the compositions is every day, or daily. In some embodiments, the treatment duration lasts until cancer remission is achieved. In some embodiments, the treatment duration is about 14 days. In some embodiments, the treatment duration is about 14 days following IR. In some embodiments, the interval of administration and treatment duration for one or more of the compositions is administration in a single, one-time dose.

In some embodiments, the mode of administration, dosage amount, interval, and treatment duration of the inhibitor of artemin pathway is intratumoral, about 10 mg/kg body weight, every other day for about 14 days following IR. In some embodiments, the mode of administration, dosage amount, interval, and treatment duration of an anti-artemin antibody is intratumoral, about 10 mg/kg body weight, every other day for about 14 days after IR. In some embodiments, the mode of administration, dosage amount, interval, and treatment duration of an anti-GFRα3 antibody (see e.g., U.S. Patent Application No. 2018/0340029A1, which is incorporated by reference) is intravenous, about 0.01-20 mg/kg body weight, administered in a single dose. In some embodiments, the mode of administration, dosage amount, interval, and treatment duration of an anti-GFRα3 antibody is intravenous, about 0.02-7 mg/kg body weight, administered in a single dose. In some embodiments, the mode of administration, dosage amount, interval, and treatment duration of an anti-GFRα3 antibody is intravenous, about 0.03-5 mg/kg body weight, administered in a single dose. In some embodiments, the mode of administration, dosage amount, interval, and treatment duration of an anti-GFRα3 antibody is intravenous, about 0.05-3 mg/kg body weight, administered in a single dose. In some embodiments, the mode of administration, dosage amount, interval, and treatment duration of the RET inhibitor is oral, about 5 mg/kg body weight, every day for about 10-20 days. In some embodiments, the mode of administration, dosage amount, interval, and treatment duration of LOXO-292 is oral, about 5 mg/kg, every day for about 10-20 days. The checkpoint inhibitors and inhibitors of the artemin pathway of the present disclosure can be in a single composition or can be in separate compositions. In some embodiments, the checkpoint inhibitor and the inhibitor of the artemin pathway are in the same composition. If the inhibitors are in separate compositions, they can be administered simultaneously or with a delay between administrations. In some embodiments, second or subsequent inhibitors can be administered 1, 2, 3, 4, 5, 6, 7, 8, 9, 10, 15, 20, 25, 30, 40, 50, or 60 minutes or longer (or any range derivable therein) after the first inhibitor is administered. In some embodiments, second or subsequent inhibitors can be administered 1, 2, 3, 4, 5, 6, 7, 8, 9, 10, 15, 20, or 24 hours or longer (or any range derivable therein) after the first inhibitor is administered. In some embodiments, subsequent inhibitors can be administered 1, 2, 3, 4, 5, 6, 7, 8, 9, 10, 15, 20, or 30 days or longer (or any range derivable therein) after the first inhibitor is administered. In some embodiments, subsequent inhibitors can be administered 1, 2, 3, 4, 5, 6, 7, 8, 9, 10, 15, 20, 25, 30, 40, or 50 weeks or longer (or any range derivable therein) after the first inhibitor is administered. In some embodiments, subsequent inhibitors can be administered 1, 2, 3, 4, 5, 6, 7, 8, 9, or 10 years or longer (or any range derivable therein) after the first inhibitor is administered.

### Methods

Methods of treating diseases are contemplated herein that utilize the therapeutic compositions and pharmaceutical compositions described herein.

The methods of the present disclosure contemplate a variety of treatment regimens. The treatment regimens contemplated can include administration of one or more radiotherapy, checkpoint inhibitor, and/or inhibitor of the artemin pathway. Each of the radiotherapy or radiotherapies, checkpoint inhibitor(s), and inhibitor(s) of the artemin pathway can be administered one or more times. In some embodiments, the compositions are administered 1, 2, 3, 4, 5, 6, 7, 8, 9, or 10 times or more.

The present disclosure contemplates sequential treatment regimens that in involve more than one therapy. In some embodiments, the treatment regimen involves one or more of radiotherapy, checkpoint inhibitor(s), and inhibitor(s) of the artemin pathway being administered “subsequent to” one or more of radiotherapy, checkpoint inhibitor(s), and inhibitor(s) of the artemin pathway. In some embodiments, “subsequent to” indicates that the subsequent treatment or group of treatments is administered subsequent to the initiation or start of the earlier treatment. In some embodiments, “subsequent to” indicates that the subsequent treatment or group of treatments is administered subsequent to the completion or final administration of the earlier treatment.

In some embodiments, the regimen comprises introducing radiotherapy to the subject before the checkpoint inhibitor and/or the inhibitor of the artemin pathway. In some embodiments of the present disclosure, the inhibitor of the artemin pathway is administered before checkpoint inhibitor(s) and/or radiotherapy. In some embodiments of the present disclosure, the checkpoint inhibitor and/or the inhibitor of the artemin pathway are administered subsequent to the radiotherapy. In some embodiments, the checkpoint inhibitor and/or the inhibitor of the artemin pathway are administered about 3-10 days subsequent to the start of the administration of the radiotherapy. In some embodiments, the inhibitor of the artemin pathway is administered subsequent to the checkpoint inhibitor and/or the radiotherapy. In some embodiments, the inhibitor of the artemin pathway is administered no more than about 7 days subsequent to the checkpoint inhibitor and/or the radiotherapy. Regimens in which two or more of radiotherapy, a checkpoint inhibitor, and an inhibitor of the artemin pathway administered simultaneously are also contemplated. In some embodiments, the inhibitor of the artemin pathway is administered simultaneously with the checkpoint inhibitor and/or the radiotherapy.

In some embodiments, the checkpoint inhibitor is administered to the subject at more than one time. In some embodiments, the checkpoint inhibitor is administered every other week. In some embodiments, the checkpoint inhibitor is administered every other week simultaneously with the radiotherapy. In some embodiments, the checkpoint inhibitor is administered every other week subsequent to the radiotherapy. In some embodiments, the inhibitor of the artemin pathway is administered to the subject at more than one time. In some embodiments, the inhibitor of the artemin pathway is administered every other day. In some embodiments, the inhibitor of the artemin pathway is administered every other day for about 14 days simultaneously with and subsequent to radiotherapy. In some embodiments, the inhibitor of the artemin pathway is administered every day. In some embodiments, the inhibitor of the artemin pathway is administered every day until remission is achieved.

The methods of the present disclosure can be used in combination with additional, distinct cancer therapies. In some embodiments, a distinct cancer therapy can include surgery, radiotherapy, chemotherapy, toxin therapy, immunotherapy, cryotherapy, and/or gene therapy. The methods of the present disclosure contemplate a variety of subject responses and endpoints for treating cancer. In some embodiments of the present disclosure, treating cancer is further defined as reducing the size of a tumor or inhibiting growth of a tumor. In some embodiments, the subject response can include reduced levels of artemin protein in tumor, spleen, and/or serum or artemin mRNA in tumor and/or spleen. In some embodiments, the subject response can include a reduced number of nodules. In some embodiments, the subject response can include a reduced number of Ter-cells in the spleen, and/or a reduced number of Ter-cells in circulation. In some embodiments, the subject response can include reduced expression of GFRα3 on tumor cells.

### Kits

As used herein, a kit may be a packaged collection of related materials, including, for example, a plurality of packages including a single and/or a plurality of dosage forms along with instructions for use.

In some embodiments, a kit includes one or more compositions in a dosage form and instructions for administering the compositions intravenously or intratumorally, or as otherwise disclosed herein. In some embodiments, the kit includes compositions comprising one or more of a checkpoint inhibitor and an inhibitor of the artemin pathway.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Study of Artemin Pathway Modulation and Cancer Therapy

### Summary

Tumor-induced CD45-Ter119+CD71+ erythroid progenitor cells (EPCs), termed “Ter-cells,” promote tumor progression by secreting artemin, a neurotropic peptide that activates RET signaling. This example demonstrates that both local tumor ionizing radiation (IR) and anti-PD-L1 treatment decreased tumor-induced Ter-cell abundance and artemin secretion outside the irradiation field and in an interferon (IFN) and CD8+ T cell-dependent manner. Recombinant erythropoietin (EPO) treatment, which was used for anemia in cancer patients who were subsequently reported to have poor outcomes of radiotherapy, promoted resistance to radiotherapy or anti-PD-L1 therapies and restored Ter-cell and artemin levels. Blockade of artemin, potential artemin signaling partners, or depletion of Ter-cells augmented the anti-tumor effects of both IR and anti-PD-L1 therapies. Analysis of samples from patients who received radio-immunotherapy demonstrated that IR-mediated reduction of Ter-cells, artemin, and one of the artemin receptors were each associated with tumor regression. Patients with melanoma who received immunotherapy had favorable outcomes associated with decreased artemin receptor levels. These findings not only demonstrate a novel out-of-field, or “abscopal” mechanism mediated by adaptive immunity that governs radiocurability, but also identify multiple targets that can improve outcomes following radiotherapy and immunotherapy.

### Introduction

Increasing interest has focused on cancer neuroscience and neurotropic ligands that support malignant progression. A population of erythroid lineage cells marked by CD45−CD71+Ter119+, termed “Ter-cells,” represents the majority of splenocytes in animals with advanced solid tumors and is associated with tumor progression in human hepatocellular carcinoma.

Tumor-induced Ter-cells secrete artemin, a neurotropic factor that belongs to the glial cell line-derived neurotropic factor (GDNF) family of ligands (GFL) whose other members include: glial cell line-derived neurotrophic factor (GDNF), neurturin (NRTN), and persephin (PSPN). These GFLs share some similar functions and signaling pathways, through receptors GFRα1-4. The ARTN homodimer binds to GFRα3-receptor as its exclusive ligand. However, in some tissues, a highly promiscuous GFRα1 receptor, which mainly associates with GDNF, is also activated by ARTN and NRTN. GFL-GFRα complexes activate the downstream proto-oncogenic trans-membrane RET (REarranged during Transfection) receptor tyrosine kinase by dimerization and phosphorylation. Following phosphorylation, RET activates multiple signaling pathways including RAS/ERK1/2, NGF/TRKA, and PI3K/AKT, pathways that mediate survival, differentiation, and proliferation of cancer cells. Under certain physiological conditions; however, RET-independent GDNF signaling has also been reported, including via neural cell adhesion molecules (NCAMs) or integrins. Recent studies have supported artemin-induced GFRα3-RET activation as a therapeutic target due to its role in promoting cell survival, tumor proliferation, metastasis, and resistance to cytotoxic therapy through possible activation of BCL2 and Twist1 pathways.

Radiation therapy is widely used in the treatment of diverse types of cancers. Recent investigations have demonstrated the importance of the immune system in mediating the anti-tumor effects of radiotherapy. Ionizing radiation (IR) mediates anti-tumor immunity through maturation of dendritic cells (DCs) and activation of T cells by enhancing DNA-sensing mediated type I/II IFN production. Investigations of immune checkpoint inhibitors, such as PD-1 inhibitors have primarily focused on enhancing T-cell function in part through increased type II IFN production. The promise of immunotherapies and combined treatments with radiotherapy warrant further research to understand the interactions between these therapies and tumor-promoting pathways.

Erythropoietin (EPO) was used in clinical trials in cancer patients receiving radiotherapy to increase red cell mass to overcome hypoxia, which is a known limiting factor in radiotherapy. Local tumor control (radio-resistance) and survival rates were not improved, however, and in some instances were worse than the control arm (not receiving EPO). These trials suggested that red blood precursor cell proliferation might be involved; and therefore, the interaction of Ter-cells, radiotherapy, and tumors was investigated. Here, it is shown that IR and anti-PD-L1 immunotherapy decreased Ter-cells and artemin levels in both pre-clinical tumor models and patients. Conversely, Ter-cells, artemin, and EPO attenuated the efficacies of both therapies. It was further determined that targeting the Ter-artemin axis enhanced the efficacy of IR and immunotherapy in model systems and that Ter-cell, artemin and artemin receptor levels are associated with outcomes in patients receiving radiotherapy, radio-immunotherapy, and immunotherapy.

### Methods

Mice. C57BL/6J wild type (WT), Ifnar1 knockout (IFNAR KO), Rag1 knockout (Rag KO), Ifng knockout (IFN-γ KO), and Ifngr1 knockout (IFNGR KO) mice were purchased from Jackson Laboratory. PD-L1 KO mice were kindly provided by L. Chen of Yale University, New Haven. All experimental groups included randomly chosen female littermates approximately 8 weeks old and of the same strain. All mice were maintained and used in accordance with guidelines established by the Institute of Animal Care and Use Committee of The University of Chicago.

Cells and reagents. MC38 and B16-SIY tumor cell lines were kindly provided by Dr. Xuanming Yang of The University of Chicago and grown in DMEM medium containing 10% FBS, at 37° C. and 5% CO₂. LLC cells were obtained from ATCC (CRL-1642). CRISPR/Cas9 was used to generate RET stable knockout MC38 cell lines, and a retrovirus overexpression system was used to generate OTI-zsGreen expressing MC38 cell line. Recombinant mouse artemin (1085-AR), EPO (959-ME), and mouse artemin antibody (AF1085) were purchased from R&D Systems. Recombinant mouse IFN-γ (315-05) was purchased from Peprotech. Depleting/blocking antibodies against PD-L1 (BE0101), CD8α (BE0004-1), IFNAR-1 (BE0241), CD4 (BP0003-1), Ter119 (BE0183), and IFN-γ (BE0055) were purchased from BioXcell. Anti-artemin was purchased from R&D (AF1085). PB-anti-CD45 (103126), FITC-anti-CD45 (103108), PE/CY7-anti-CD71 (113812), PE-anti-CD71 (113808), APC-anti-Ter119 (116212), PE-anti-H-2Kb (116507), PE-anti-CD4 (116005), and APC/CY7-anti-CD8 (100714) were purchased from Biolegend. AF488-anti-GFRα3 (SC-398618 AF488) was purchased from SantaCruz. LOXO-292 (C-1911) was purchased from Chemgood. CD8 T cell selection kit (18953) was purchased from Stemcell, and CD45 selection kit (8802-6865-74) was purchased from Thermo Fisher Scientific®.

Tumor models and treatments. 1×10⁶ MC38, LLC, or B16-SIY tumor cells were subcutaneously injected into the flank of mice. On day 10 after tumor inoculation, tumors were either irradiated with one dose of 20 Gy or mice received sham treatment. For anti-PD-L1 treatment experiments, 200 μg anti-PD-L1 (10F.9G2) or isotype control was given by i.p. every three days for a total of four times starting on day 10 after tumor inoculation. For type I IFN blockade experiments, 200 μg anti-IFNAR1 were intratumorally injected on days 0 and 2 after irradiation. For CD4 or CD8 T cell depletion experiments, 200 μg anti-CD4 or anti-CD8 mAb was delivered four times by i.p. injection every 3 days starting 1 day before therapies. Artemin-neutralizing antibody was delivered i.t. at 1 μg/mouse starting on day of irradiation, every 2 days for 7 doses. For Ter-cell depletion, anti-ter119 was injected i.p. at 20 μg/mouse every 2 days for 4 doses. For Ter-cell or artemin treatment groups, mice were administered 1×10⁷ purified Ter cells i.v. every other day for a total of three doses, or mice were treated with 0.5 μg/mouse artemin i.t. every other day starting on day 0 of therapies throughout the studies. For EPO treatment groups, mice were treated with 20 U/mouse EPO i.v. every other day throughout the studies, starting on day 0 of the therapies. IFN-γ was administered through intrasplenic injection on day 15 post tumor implantation at 2 μg/mouse for 1 dose. LOXO-292 was administered by oral gavaging at 100 μg/mouse/day throughout the entire studies, starting on day 0 of the therapies. Spleens were harvested on day 20 or at indicated times post-inoculation for analysis of spleen size or splenic cells. Tumor size was monitored and calculated with the formula for area (length×width).

Flow Cytometry. Tumor and lung tissues were cut into small pieces and digested by 1 mg/mL collagenase IV (Sigma) and 0.2 mg/mL DNase I (Sigma) for 1 hr at 37° C. Spleens, lymph nodes, and bone marrow were ground prior to analysis. Single cell suspensions were blocked with anti-FcR (2.4G2, BioXcell) and then stained with fluorescence-labeled antibodies. Flow cytometry was performed on BD LSR Fortessa at The University of Chicago core facility and data were analyzed with FlowJo software.

ELISA. Tumor tissues were homogenized in PBS with protease inhibitor followed by the addition of Triton X-100. Serum was collected on day 20 post-tumor inoculation or at indicated times. The concentration of artemin or TGF-β was measured with artemin ELISA Kit (E03A0032 for mouse and E01A0032 for human, BlueGene Biotechnology), or TGF-β1 Mouse ELISA Kit (BMS608-4, Invitrogen).

Western Blot Analysis. Whole-cell protein was extracted with Triton-X100 buffer (150 mM sodium chloride, 50 mM Tris, 1% Triton-X100; pH 8.0) with proteinase inhibitors (Thermo Scientific®). Immuno-blotting analyses were performed as previously described (Hou, Y., Liang, H., Rao, E., Zheng, W., Huang, X., Deng, L., Zhang, Y., Yu, X., Xu, M., Mauceri, H., et al. (2018). Non-canonical NF-kappaB Antagonizes STING Sensor-Mediated DNA Sensing in Radiotherapy. Immunity 49, 490-503 e494). The amount of loaded protein was normalized to GAPDH (60004-1-Ig, Proteintech Group) or actin (8226, Abcam).

Real-time PCR assay. mRNA from tumor cells or splenocytes was isolated using TRIzol according to the manufacturer's instructions (Invitrogen). cDNA was synthesized from pd(N)6-primed mRNA reverse transcription using M-MLV superscript reverse transcriptase. Real-time PCR kits (SYBR Premix Ex Taq™, DRR041A) were purchased from Takara Bio Inc. PCR was performed using a CFX96 (Bio-Rad). mRNA specific for the housekeeping gene GAPDH was measured and used as an internal control. The primers for artemin were 5′-TAC TGC ATT GTC CCA CTG CCT CC-3′ (SEQ ID NO: 1) for the upstream primer (UP) and 5′-TCG CAG GGT TCT TTC GCT GCA CA-3′ (SEQ ID NO: 2) for the downstream primer (DP); GAPDH: 5′-AGA CCA GCC TGA GCA AAA GA-3′ (SEQ ID NO: 3) for UP and 5′-CTA GGC TGG AGT GCA GTG GT-3′ (SEQ ID NO: 4) for DP.

Statistical analysis. Analyses were performed using GraphPad Prism software 6. Data were analyzed by one-way ANOVA with Multiple Comparison Test or Student's t-test. P values <0.05 were considered statistically significant.

### Results

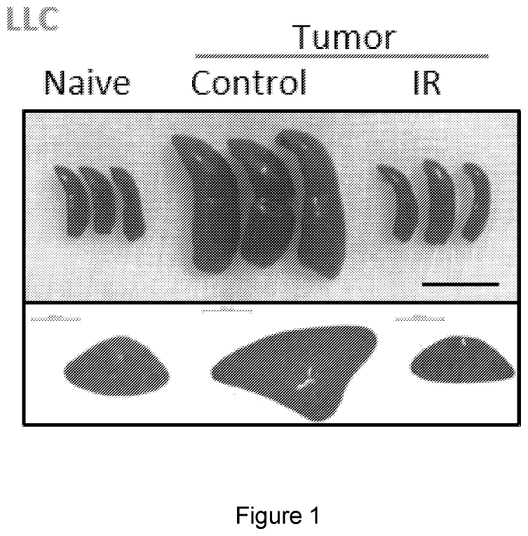

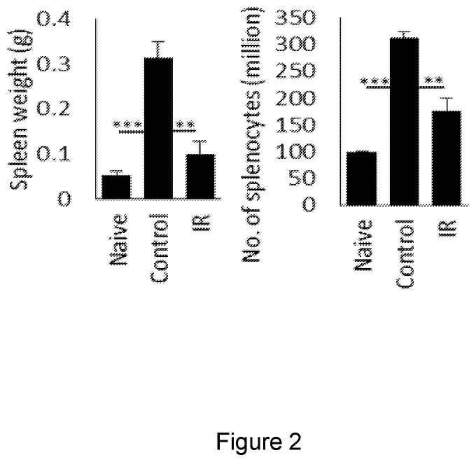

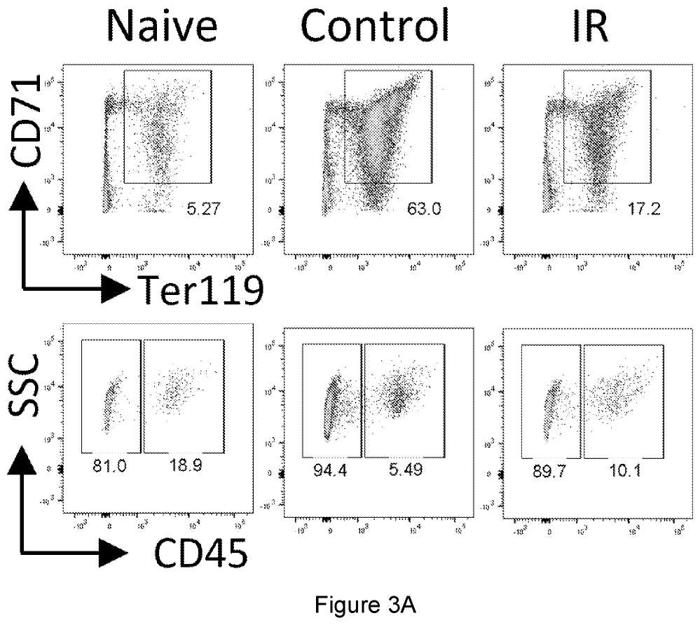

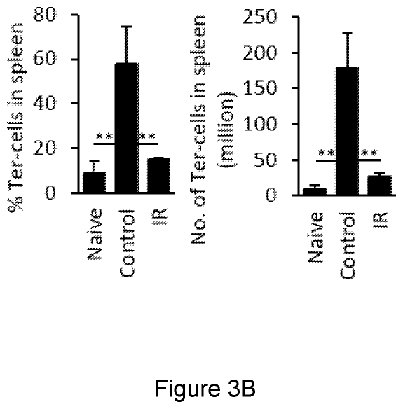

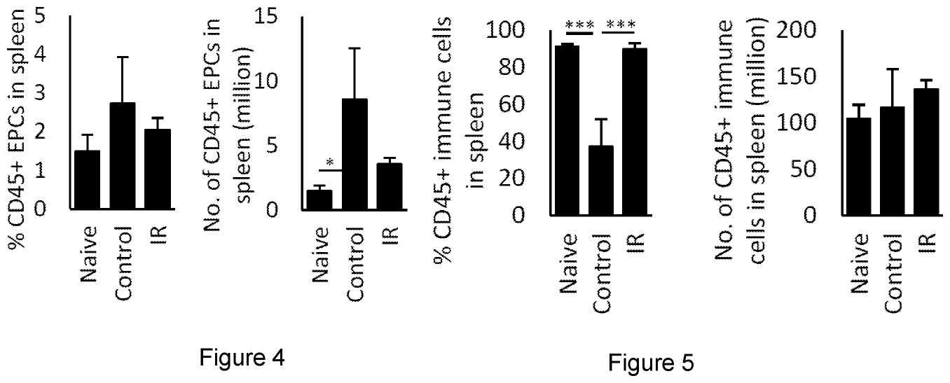

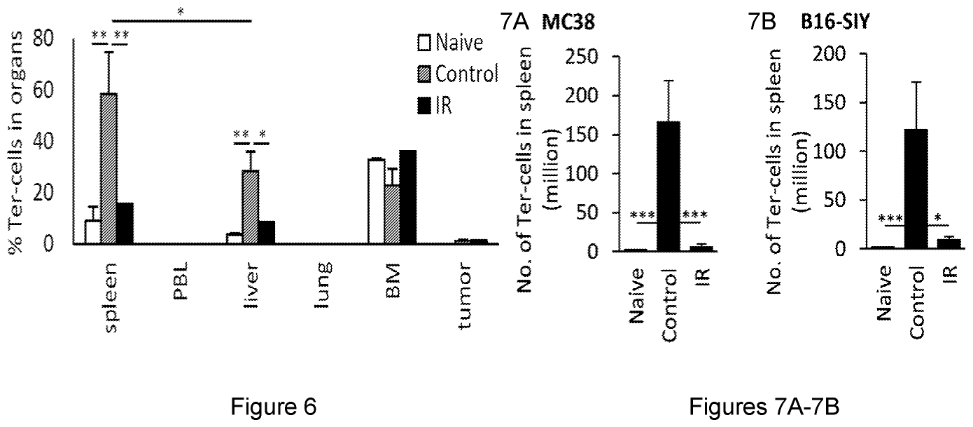

Local irradiation decreases tumor-induced Ter-cell accumulation in the murine spleen. The observations that tumor-induced erythroid progenitor cells (EPCs) correlate with a poor prognosis suggest that tumor progression might be inhibited by targeting EPCs or their secretable products. It was observed initially that tumor-bearing mice developed splenomegaly, and that spleen size normalized following irradiation of flank tumors (FIG. 1). To determine whether radiotherapy affects tumor-induced EPCs, Lewis Lung cancers (LLC) inoculated into the flanks of syngeneic mice were treated with local ionizing radiation (IR) while shielding the rest of the body and quantified EPCs in various organs of both control and tumor-bearing mice. Tumor-induced splenomegaly was dramatically decreased following IR (FIG. 1), with spleens returning to baseline size. The splenic weight and number of splenocytes in tumor-bearing mice were decreased by IR (FIG. 2). Previously, two population of EPCs in the spleen were characterized that increased by large numbers in tumor-bearing mice and in late-stage cancer patients: notably, CD45− and CD45+ EPCs. It was observed that most EPCs in mouse spleen were CD45− (Ter-cells) (FIGS. 3A, 3B). Ter119⁺CD71⁺ EPCs accounted for the largest percentage of the splenocytes that were decreased by IR. By contrast, the number of CD45⁺ EPCs was unchanged following IR (FIG. 4). Since CD45⁺ immune cells and CD45⁺ EPCs were unchanged by IR (FIG. 5), IR-induced reduction of spleen size was predominantly due to a reduction in the number of Ter-cells. Ter-cells were previously reported to populate the spleen preferentially; however, in the LLC tumor model, Ter-cells were also present in the liver of tumor-bearing mice, although the initial percentage of Ter-cells in the liver was lower than in the spleen (28% vs. 58%). IR decreased tumor-induced Ter-cells in the liver, as well (FIG. 6). In addition, similar Ter-cell reductions in other tumor models, such as MC38 colon cancer and B16-SIY melanoma, was detected following IR (FIGS. 7A, 7B), which suggest that IR-induced reduction of Ter-cells was not tumor-type dependent.

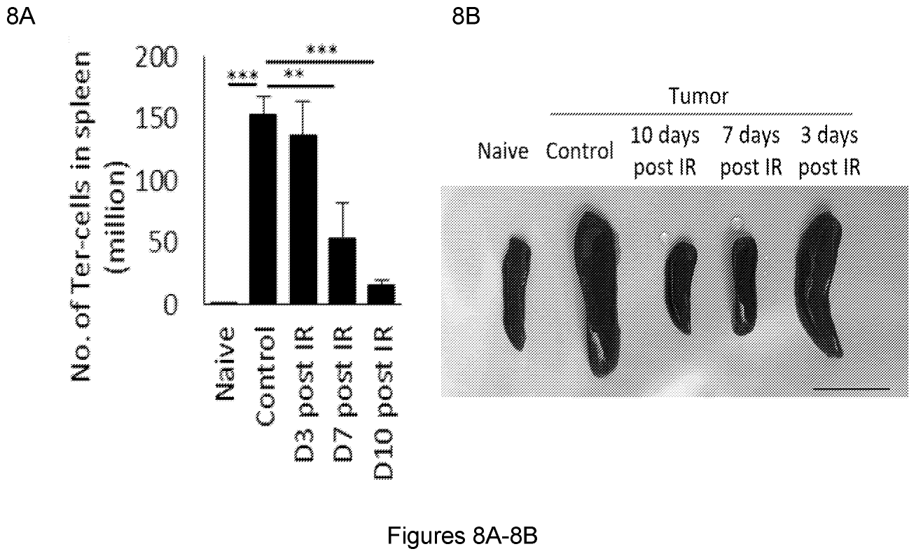

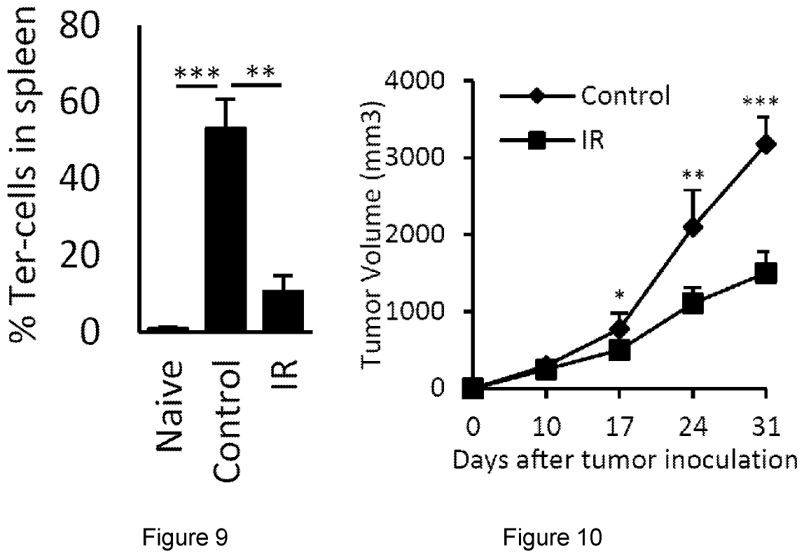

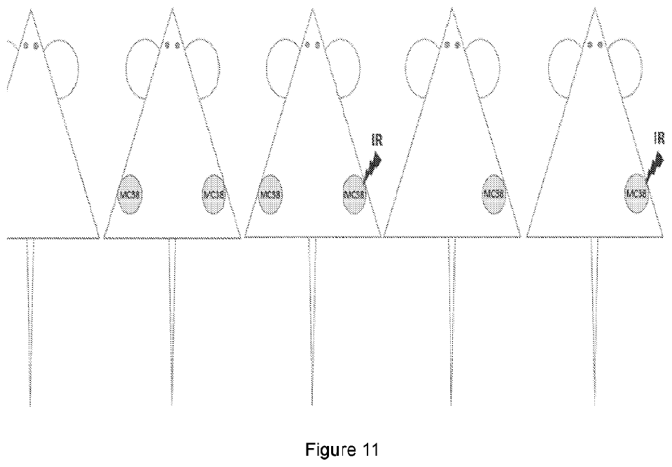

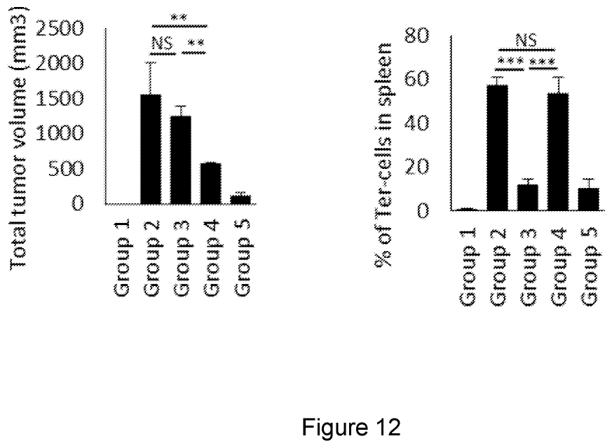

To characterize the process by which IR modulates Ter-cell abundance in the spleen, the kinetics of IR-mediated Ter-cell reduction was assessed, and it was determined that the number of Ter-cells began decreasing 7 days following IR, reaching complete normalization to baseline size at day 10 (FIGS. 8A, 8B); this effect persisted for at least 20 days (FIG. 9). To address whether the reduction in Ter-cell number was dependent on a corresponding reduction of tumor burden by IR (FIG. 10), a dual tumor model was designed, in which 5 groups of mice were investigated: (1) no tumor, (2) two tumors not irradiated, (3) two tumors with 1 tumor receiving IR, (4) one tumor not irradiated, and (5) one tumor receiving IR (FIG. 11). At day 10 following IR, the total tumor volume of group 3 (two tumors with one receiving IR) was comparable with that of group 2 (two tumors not irradiated), but the splenic Ter-cell abundance in group 3 was significantly lower than that in group 2. In addition, the total tumor volume of group 4 (one tumor, not irradiated) was lower than that in groups 2 and 3, but the number of Ter-cells was comparable with that in group 2 and higher than that in group 3 (FIG. 12). These results demonstrated that the observed reduction of Ter-cells in the spleen was due to the effects of radiation treatment rather than reduction in tumor burden.

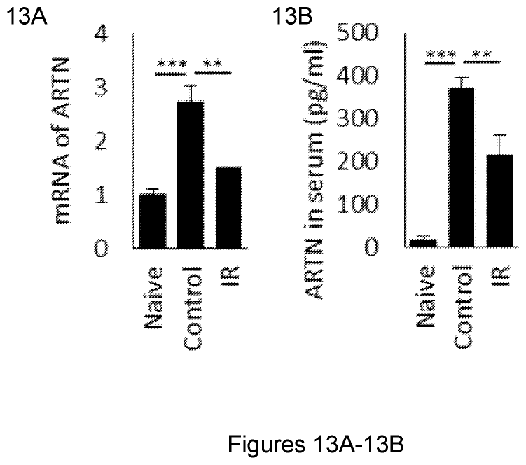

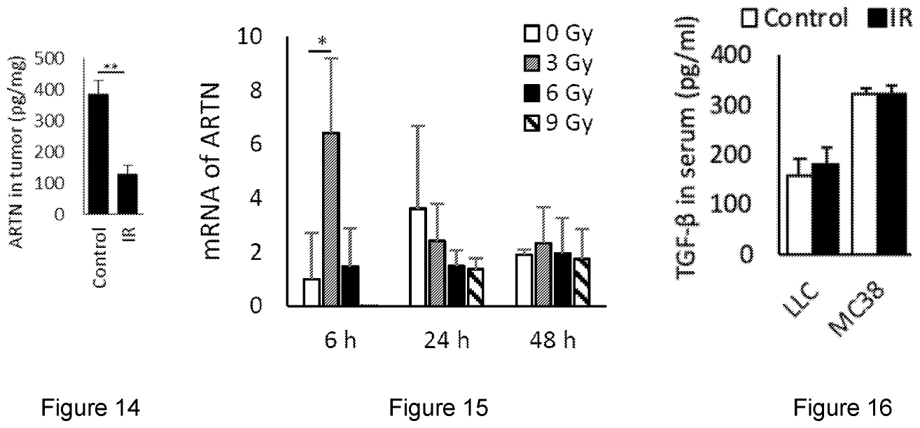

Ter-cells promote tumor progression by secreting artemin, an oncogenic factor associated with chemo- and radio-resistance. Artemin expression following IR was examined, and it was determined that IR decreased both artemin mRNA expression in the mouse spleen and protein levels in the serum (FIGS. 13A, 13B). In addition, IR suppressed total artemin protein levels in the tumor microenvironment (FIG. 14) despite IR induction of mRNA levels in tumor cells (FIG. 15). These results suggest that both tumoral and circulating artemin is, in large part, derived from Ter-cells and not tumor cells; local tumor irradiation decreases splenic Ter-cells (via a remote effect) and as a result reduces artemin secretion.

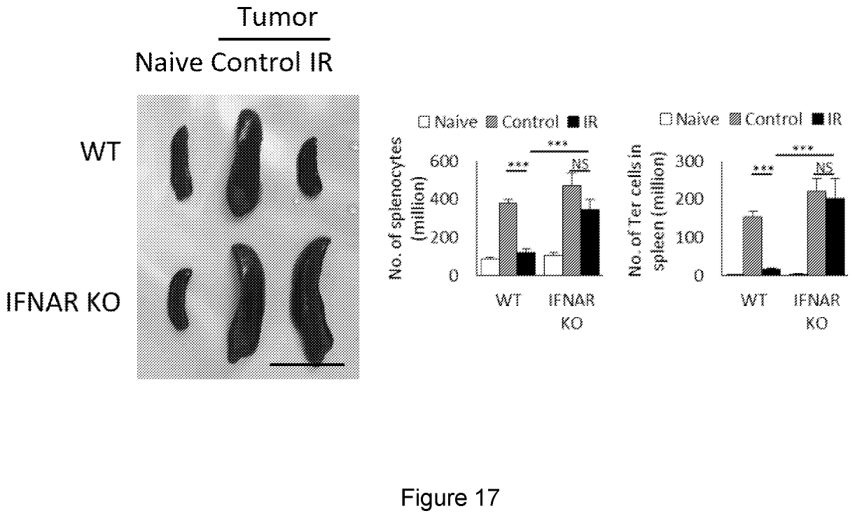

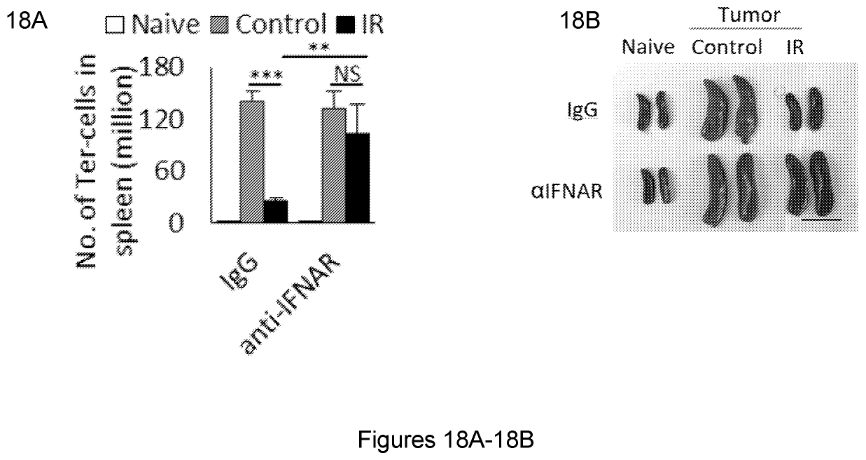

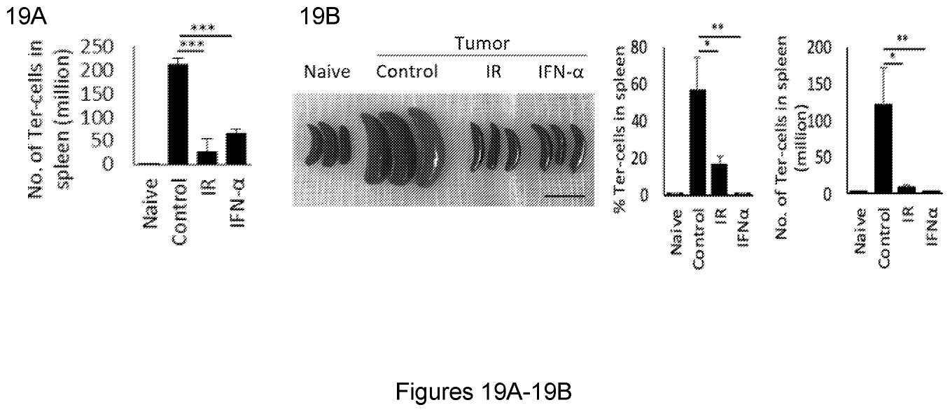

IFNs and T cells are required for the effect of irradiation on Ter-cells. The mechanism by which local tumor irradiation decreased Ter-cell accumulation in the mouse spleen was explored. Although it has been reported that tumor-derived TGF-β mediates the generation of Ter-cells in the spleen, it was determined that irradiation of local tumors did not decrease TGF-β levels in the serum (FIG. 16). Irradiation triggers local and systemic anti-tumor immunity via type I interferons (IFNs) and T cells. To determine the potential role for type I IFNs in Ter-cell reduction, tumors were established in interferon alpha receptor knock-out mice (IFNAR KO). Spleen size, splenocyte number, and Ter-cell number were decreased by IR in wild-type mice (WT), but not in IFNAR KO mice (FIG. 17), which suggested that host type I IFN signaling was required for the effect of IR on Ter-cells. To exclude any possible genetic defect of transgenic mice that might confound the results, tumor-bearing WT mice were treated with IR and/or IFNAR antibody to block mouse IFNAR. The results showed that IR did not decrease Ter-cells in presence of IFNAR antibody (FIGS. 18A, 18B). To further confirm the role of type I IFNs on Ter-cells, tumors in WT mice were treated with either local IR or exogenous IFN-α through intra-tumor injection (i.t.), and both IR and IFN-α reduced Ter-cell accumulation in the spleen of mice bearing LLC or B16-SIY tumors (FIGS. 19A, 19B). Thus, type I IFNs are required and sufficient for IR-mediated Ter-cell reduction.

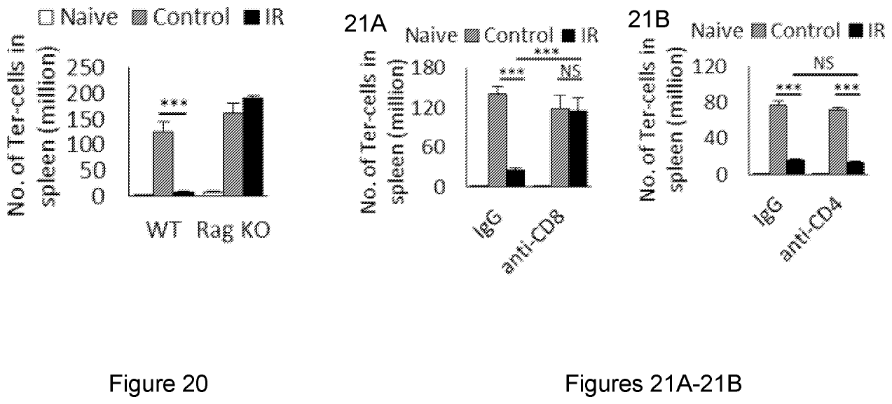

Type I IFNs promote T cell responses by both enhancing the function of antigen presenting cells (APCs) to process and present antigens and promoting the survival of T cells. Given that T cells are in part responsible for the systemic effects of IR and are abundant in the spleen, the role of T cells in Ter-cell reduction was examined. IR did not decrease Ter-cell abundance in Rag1 KO mice, in which T cells are deficient (FIG. 20). This result suggested that T cells are required for the observed IR-induced Ter-cell reduction. To study which T cell subsets are required, either CD4⁺ or CD8⁺ T cells were depleted using blocking antibodies. The results showed that depletion of CD8⁺ T cells, but not CD4⁺ T cells, abrogated IR-induced Ter-cell reduction (FIGS. 21A, 21B).

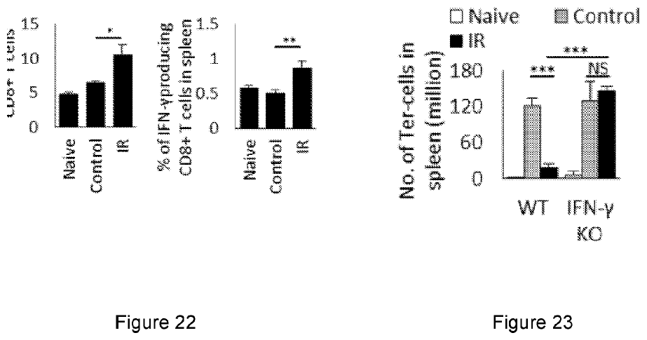

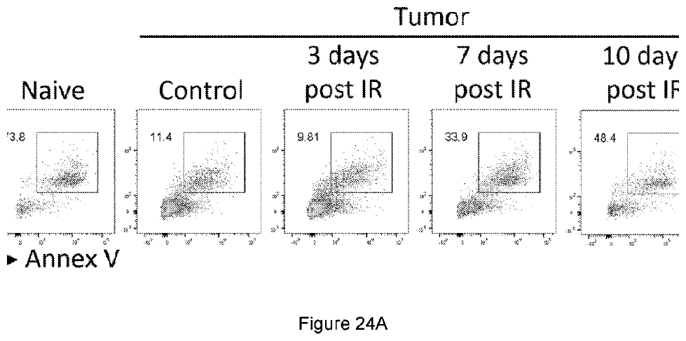

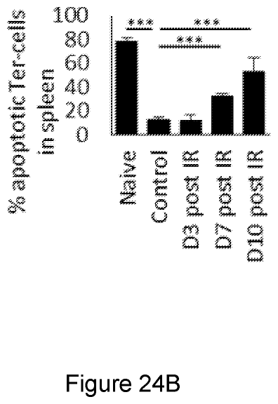

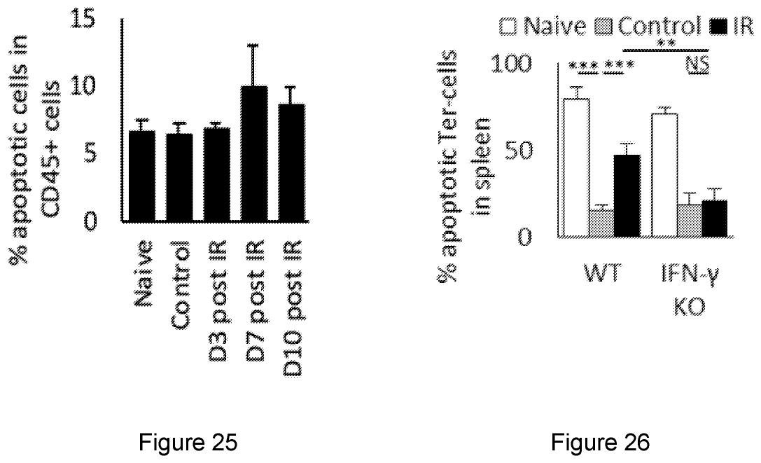

Having established that CD8⁺ T cells play a role in the IR-induced reduction of Ter-cells, factors produced by these CD8⁺ cells were investigated. It was found that IR increased IFN-γ production in CD8⁺ T cells in the spleen (FIG. 22). Therefore, the role for IFN-γ on Ter-cell reduction following IR was investigated. By using IFN-γ KO mice, it was found that IFN-γ was also required for IR-induced Ter-cell reduction (FIG. 23). Because IFN-γ mediates apoptosis of erythrocytes, the fraction of Ter-cells undergoing apoptosis was explored. It was found to be approximately 70% in naive mice and approximately 10% in tumor-bearing mice. This finding is consistent with the observation that a large tumor burden increases Ter-cell number. Importantly, IR restored high apoptosis levels of Ter-cells in tumor-bearing mice (FIG. 24) 7 days post treatment, coincident with the reduction of Ter-cell numbers following IR (FIG. 8A). In contrast, the level of apoptosis of CD45⁺ immune cells at baseline was lower than that of CD45-Ter-cells, and it remained unchanged by either tumor-bearing or IR treatment (FIG. 25). The results demonstrate Ter-cell apoptosis in IFN-γ KO tumor bearing mice is not restored by IR (FIG. 26); the results therefore suggest that IFN-γ is required for IR-induced Ter-cell apoptosis.

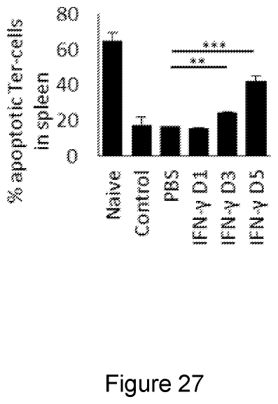

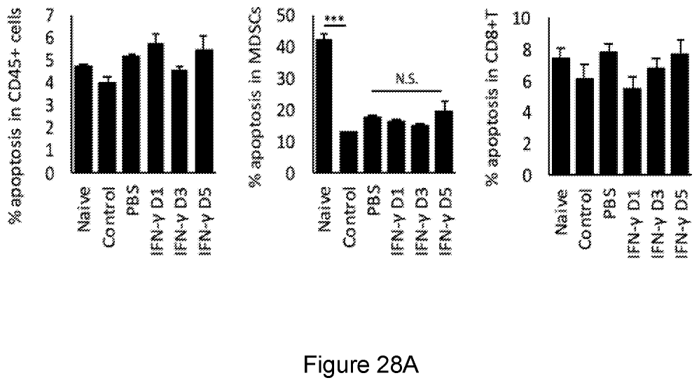

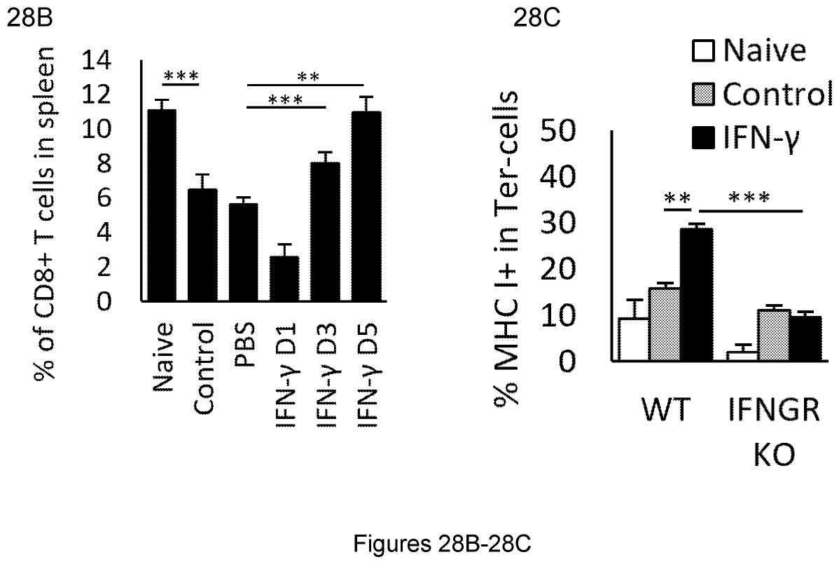

To further assess the role of IFN-γ, tumor-bearing mice were treated with exogenous IFN-γ through intra-splenic injection, and it was determined that IFN-γ increased apoptosis of Ter-cells 3 days after treatment (FIG. 27). By contrast, IFN-γ did not change apoptosis levels of immune cells, including MDSCs and CD8⁺ T cells (FIG. 28A). Therefore, IFN-γ was required and sufficient to induce Ter-cell apoptosis. In spleens treated with IFN-γ, it was observed that IFN-γ restored the abundance of CD8⁺ T cells that decreased in spleens of tumor-bearing mice (FIG. 28B). In addition, IFN-γ enhanced the MHC I expression on Ter-cells (FIG. 28C). These data suggest that IFN-γ promotes the apoptosis of Ter-cells by increasing CD8 T⁺ cell abundance in spleen and raises the potential of both direct killing of Ter-cells by gamma interferon and/or T cell MHC I specific killing of Ter-cells. Taken together, the data demonstrate that local irradiation reduces tumor-induced Ter-cell accumulation in spleen in a type I/II IFN and CD8⁺ T cell dependent manner.

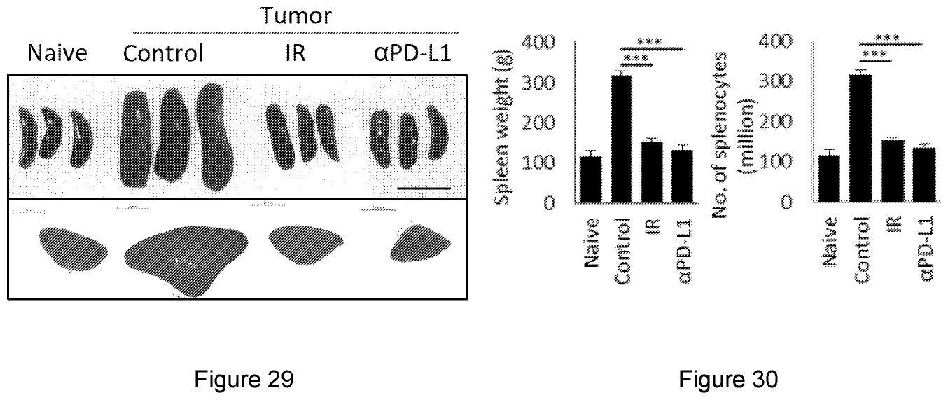

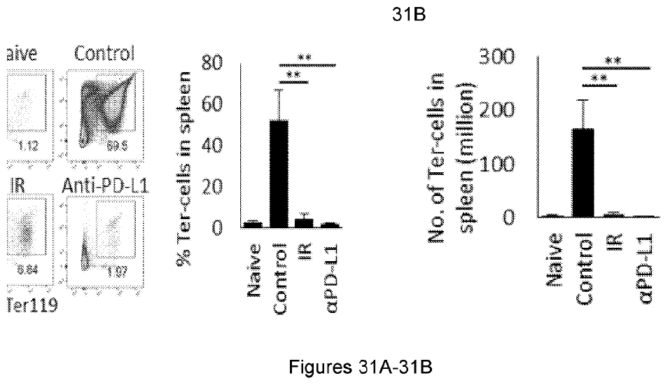

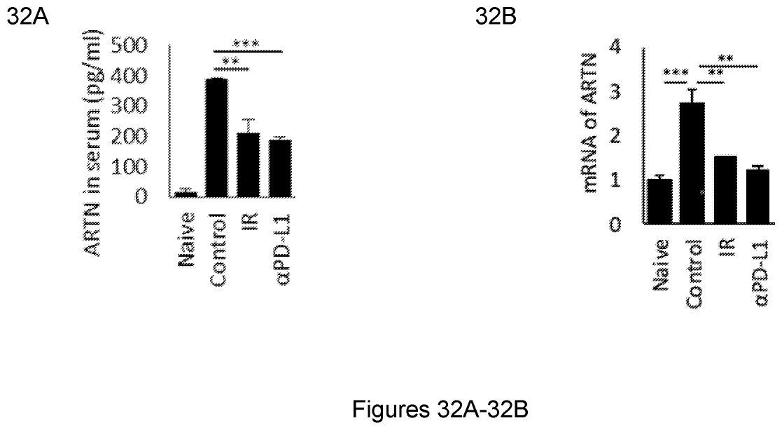

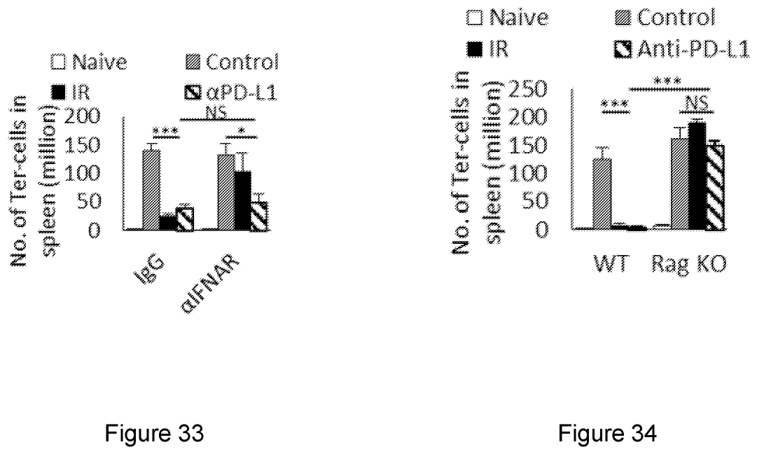

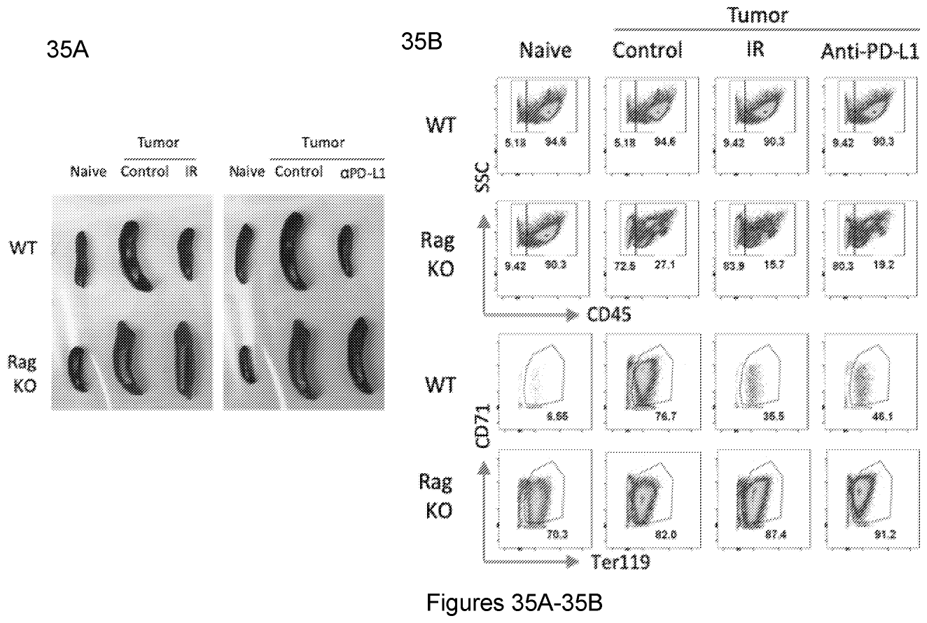

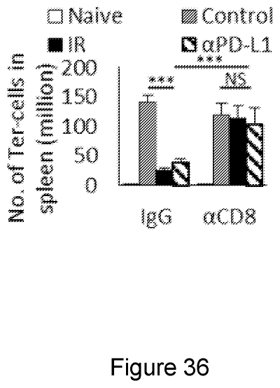

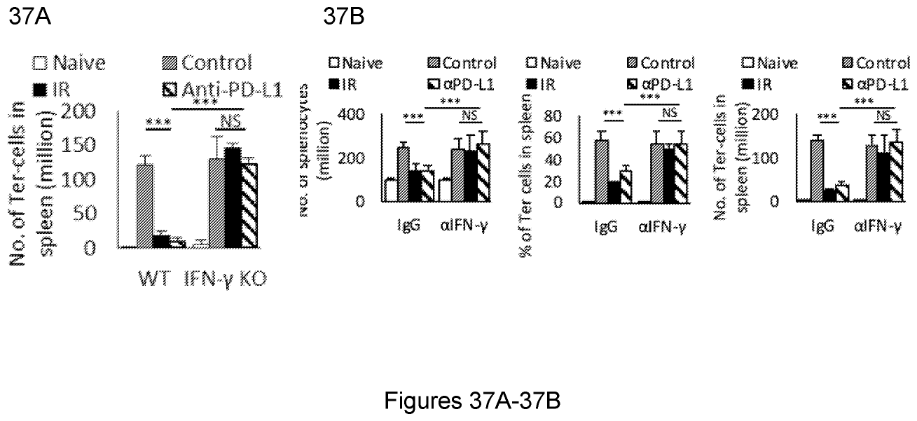

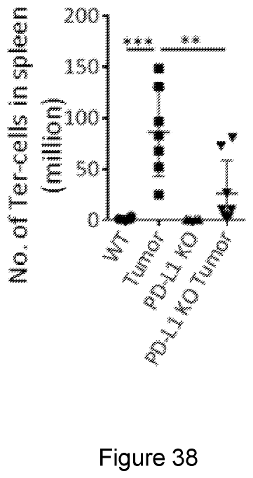

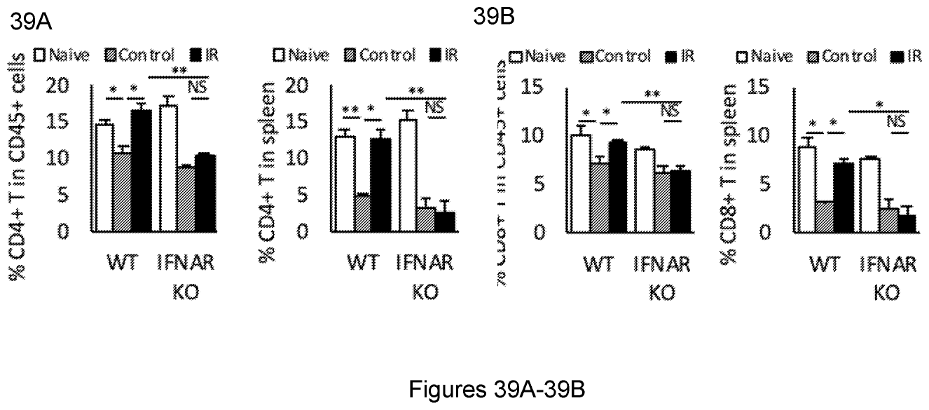

PD-L1 blockade reduces tumor-induced Ter-cell accumulation in a CD8⁺ T cell- and IFN-γ dependent manner. Immunotherapies, including PD-L1/PD-1 blockade, are promising treatments for many cancers. PD-L1/PD-1 blockade enhances the immune functions of CD8⁺ T cells, including IFN-γ production and cytotoxic activity. It was hypothesized that PD-L1 blockade might control tumor-induced splenic Ter-cell accumulation by a similar mechanism as radiation which is reported to induce T cell priming. Tumor-bearing mice were treated with either intraperitoneal administration of PD-L1 blocking antibody (αPD-L1) or IR and found that each treatment decreased tumor-associated splenomegaly (FIG. 29) and the number of splenocytes (FIG. 30). Flow cytometric analysis showed that PD-L1 blockade reduced the percentage and number of Ter-cells in the spleen of tumor-bearing mice (FIGS. 31A, 31B). As expected, PD-L1 blockade also decreased artemin expression in the spleen and artemin protein levels in serum (FIGS. 32A, 32B). The next experiments were to determine whether PD-L1 blockade regulated Ter-cell accumulation through type I IFNs, CD8⁺ T cells, and IFN-γ. As shown in FIG. 33, PD-L1 blockade decreased the number of Ter-cells in the spleen with and without IFNAR blocking antibody treatment. These results suggest that type I IFN signaling is not required for the Ter-cell killing action of PD-L1 blockade. In contrast, splenic Ter-cells were not decreased by PD-L1 blockade in Rag KO mice (FIGS. 34, 35A, and 35B). Depletion assays demonstrated that CD8⁺ T cells were required for anti-PD-L1 mediated Ter-cell reduction (FIG. 36). By using IFN-γ KO mice and IFN-γ neutralizing antibody, it was found that PD-L1 blockade decreased the number of Ter-cells in an IFN-γ dependent manner (FIGS. 37A, 37B). Consistent with these results using PD-L1 blockade, the levels of tumor-induced splenic Ter-cells in PD-L1 KO mice were lower than those in WT mice (FIG. 38). These data suggested that PD-L1 blockade bypasses type I IFN signaling and acts directly on T cells to control tumor-induced Ter-cell abundance in the spleen. In contrast to anti-PD-L1 treatment, type I IFN signaling was required for the IR-mediated Ter-cell reduction (FIGS. 39A, 39B), suggesting that type I IFN signaling played a central role in the IR-induced systemic T cell response required for Ter-cell elimination in tumor-bearing mice. Thus, PD-L1 blockade decreased tumor-induced Ter-cell accumulation as well as artemin production.

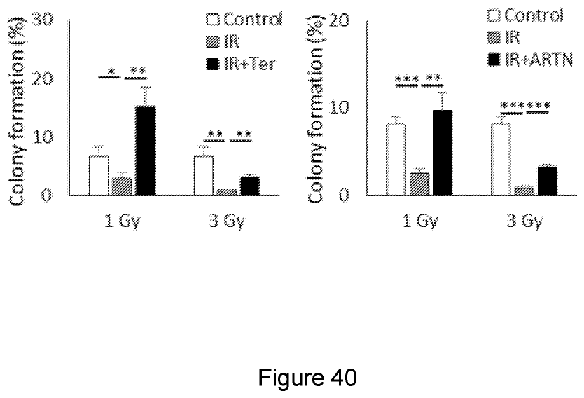

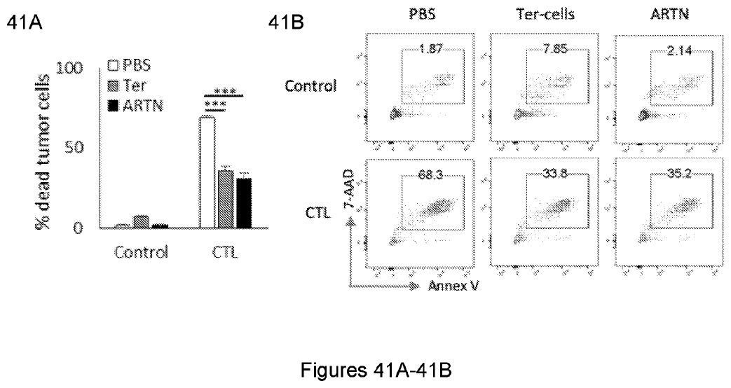

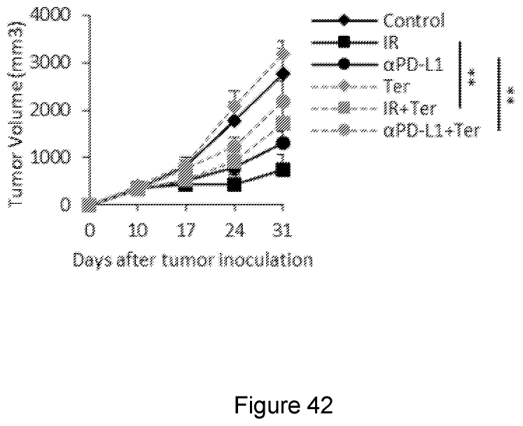

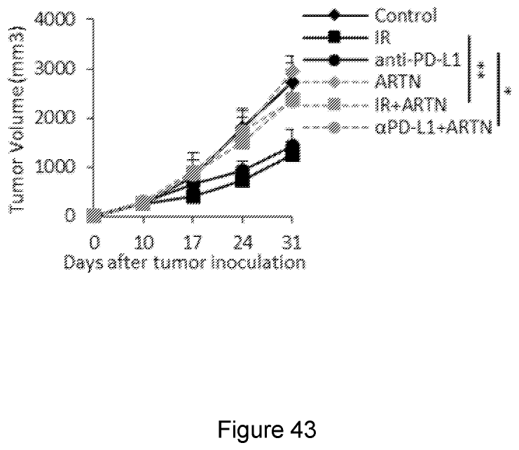

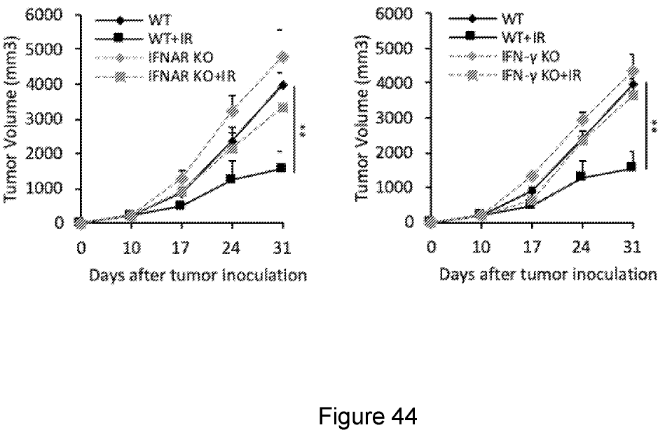

Ter-cells and artemin impair the therapeutic effect of radio- and immunotherapy. Next, the role of Ter-cells and artemin on the therapeutic effects of IR and PD-L1 blockade was investigated. Using the colony formation assay, it was found that Ter-cells and artemin increased the radio-resistance of mouse MC38 tumor cells (FIG. 40), which was consistent with the role of artemin in human MDA-MB-231 and BT549 epithelial carcinoma cells. It was determined that Ter-cells and artemin impaired the CD8⁺ T cell mediated killing of tumor cells (FIGS. 41A, 41B). These data suggest that Ter-cells and artemin inhibit T-cell cytotoxic killing ability. Adoptive transfer of Ter-cells or administration of exogenous artemin diminished the anti-tumor effects of IR and PD-L1 blockade (FIGS. 42, 43). It was also found that IR failed to control tumor growth in either IFNAR KO mice or IFN-γ KO mice, in which tumor-induced Ter-cells were not decreased by IR (FIG. 44). These data suggest that Ter-cells and artemin impaired the effects of both IR and immunotherapy. These results considered with reduction of Ter-cells and artemin by IR and anti-PD-L1 suggest that the full therapeutic effect of IR and immunotherapy partially depends on their suppressive effect on Ter-cells.

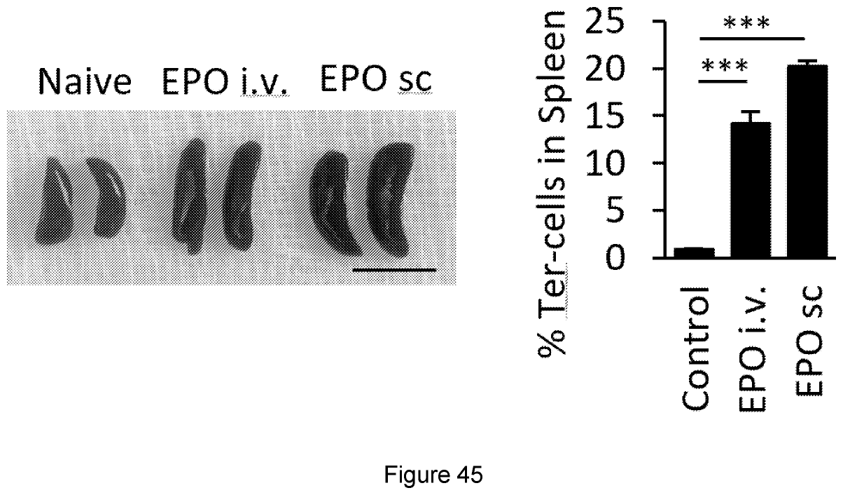

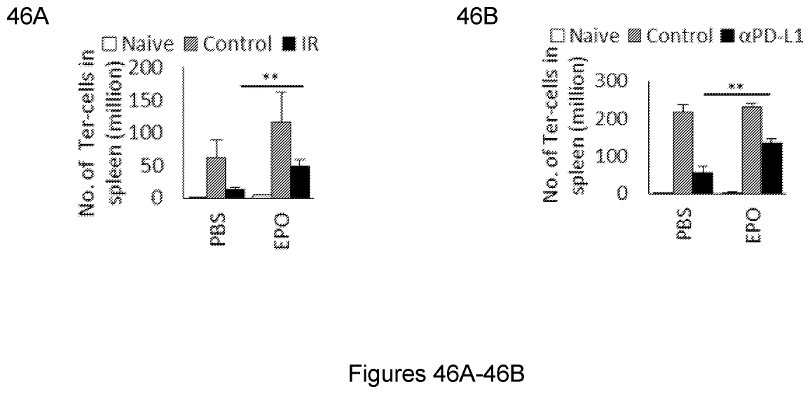

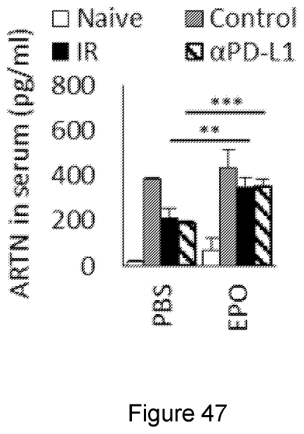

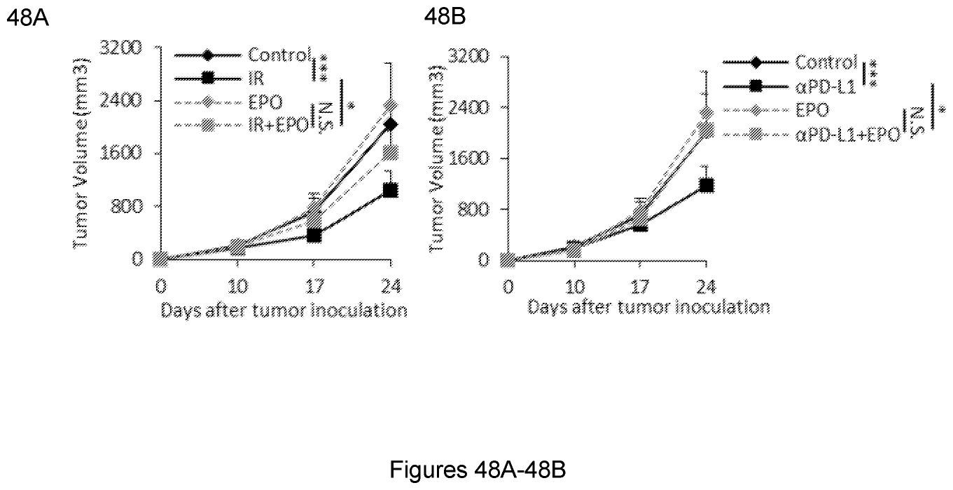

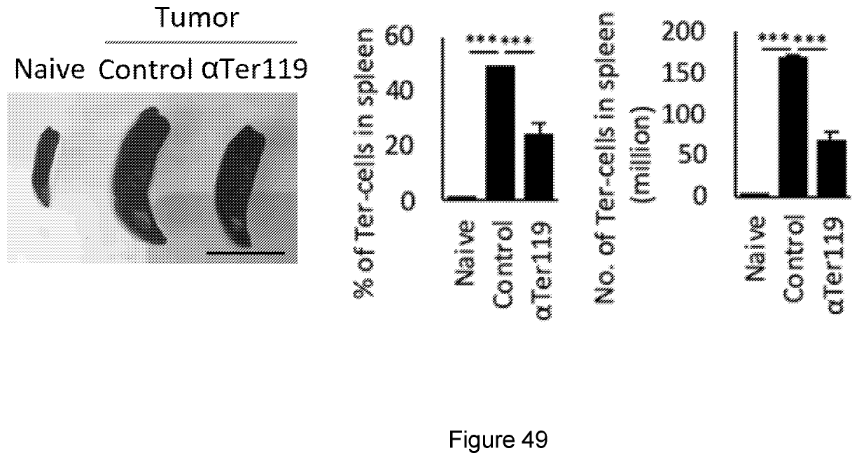

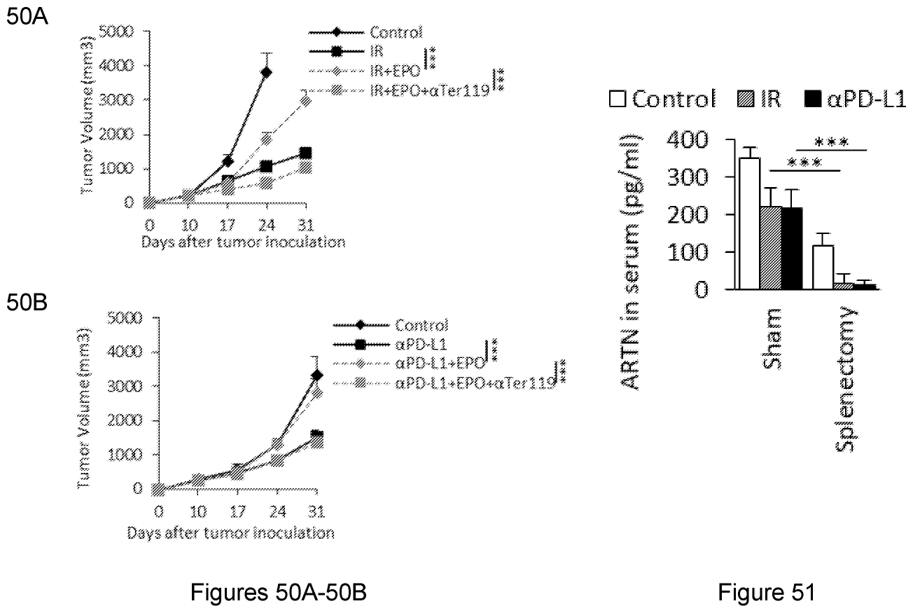

Recombinant human erythropoietin (rhEPO) has been used for the treatment of anemia in cancer patients during chemotherapy or radiotherapy. Several clinical trials have reported on the detrimental effects of rhEPO on survival benefit in cancer patients treated by RT via unknown mechanisms. To determine whether EPO impairs the effect of radiotherapy indirectly by enhancing Ter-cell production and artemin secretion, it was first determined whether rmEPO increased Ter-cells in mouse spleen. The results indicate that administration of EPO through i.v. and s.c. routes increased spleen size and Ter-cell accumulation in naive mice (FIG. 45). Additionally, EPO diminished the effects of IR and PD-L1 blockade on Ter-cell accumulation (FIGS. 46A, 46B), as well as the level of secreted artemin in the serum (FIG. 47). Consistent with these findings, the administration of EPO inhibited the therapeutic effect of both IR and PD-L1 blockade (FIGS. 48A, 48B). To further confirm that EPO inhibited the therapeutic effects of both IR and PD-L1 blockade by increasing Ter-cell production, mice were treated with Ter119 antibody, which resulted in depletion of tumor-induced Ter-cells (FIG. 49). The result showed that Ter119 depleting antibody in the context of EPO treatment restored the therapeutic effects of IR and PD-L1 blockade (FIGS. 50A, 50B). Thus, EPO was detrimental to the anti-tumor effects elicited by both IR and PD-L1 blockade via its augmenting effect on Ter-cells.

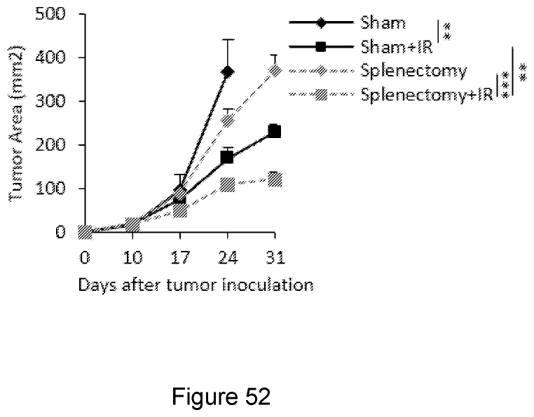

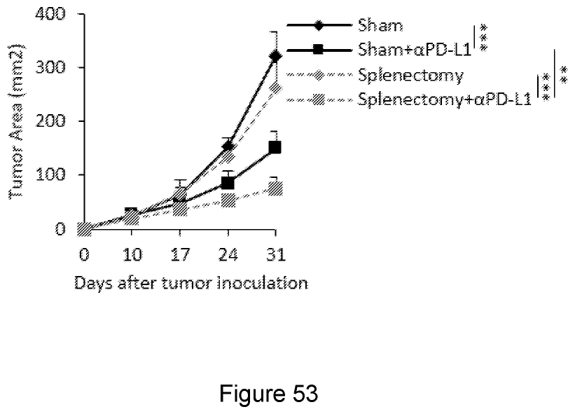

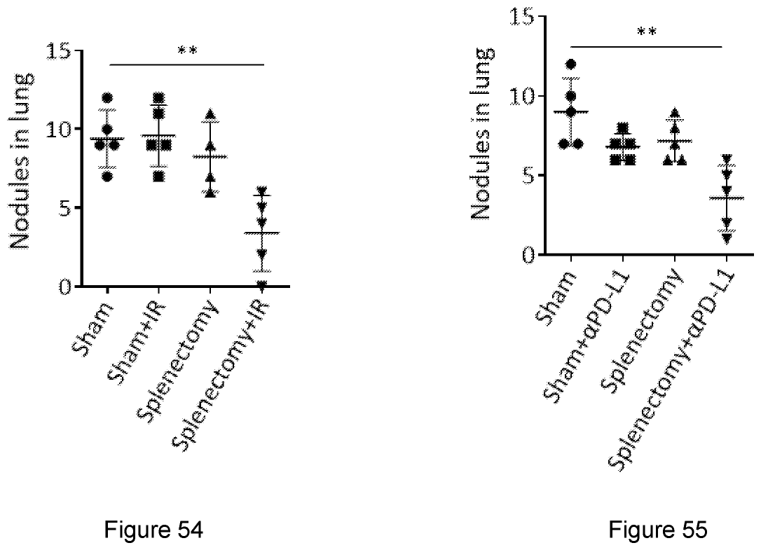

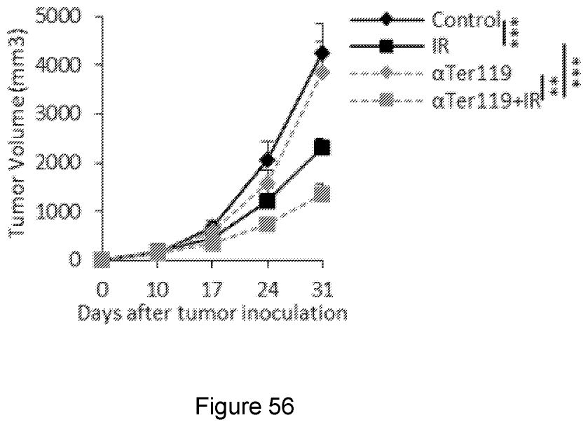

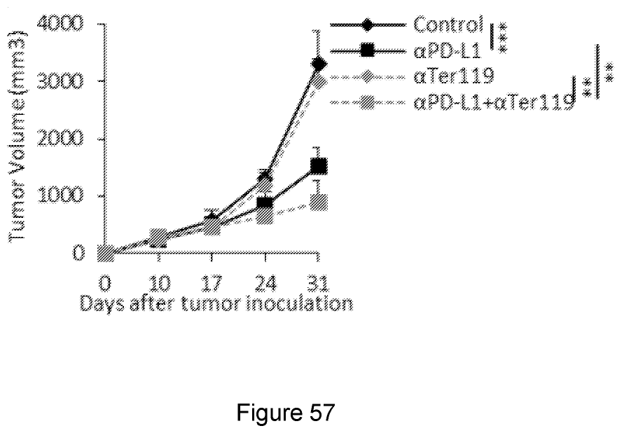

Disrupting the Ter-cell/artemin axis promotes the therapeutic effect of both RT and immunotherapy. Ter-cells impair the therapeutic effects of IR and PD-L1 blockade through artemin secretion (FIGS. 42, 43). However, neither IR nor PD-L1 blockade completely abrogated elevated artemin levels in the serum of tumor-bearing mice (FIG. 32A), although these treatments reduced Ter-cell levels to levels similar to non-tumor bearing mice (FIGS. 31A, 31B). The next experiments were designed to determine whether the therapeutic effects of IR and PD-L1 blockade could be improved by targeting the Ter-cell/artemin axis. It was found that splenectomy produced synergistic effects with both IR and PD-L1 blockade on reducing artemin levels (FIG. 51). Splenectomy also enhanced the therapeutic effect of IR (FIG. 52) and PD-L1 blockade (FIG. 53). Interestingly, it was found that neither IR nor splenectomy alone affected the spontaneous metastasis of LLC tumor, but the combination treatment significantly decreased pulmonary metastases (FIG. 54). Consistent with these findings, the combination treatment of PD-L1 blockade and splenectomy decreased pulmonary metastases (FIG. 55), although treatment with PD-L1 blockade alone showed minimal inhibition of metastasis. By using anti-Ter119, it was determined that depleting Ter-cells also promoted the effect of IR (FIG. 56) and PD-L1 blockade (FIG. 57).

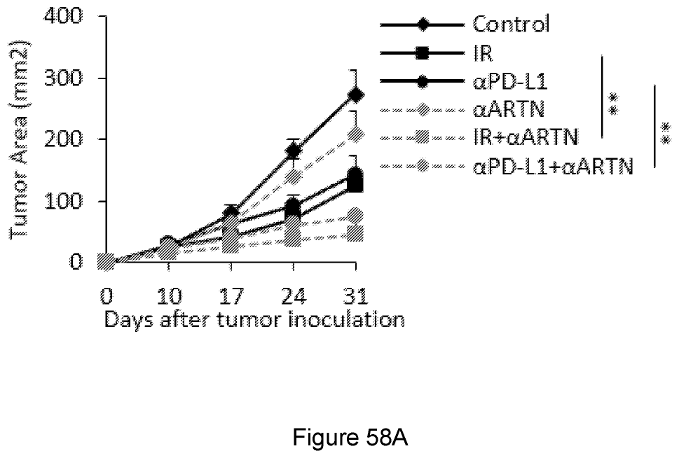

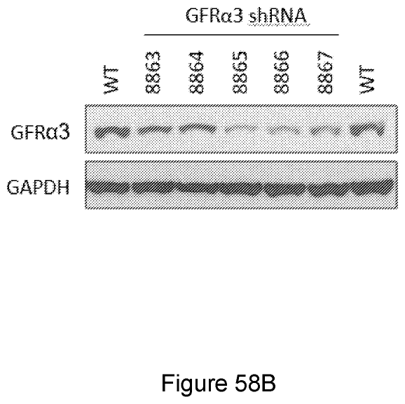

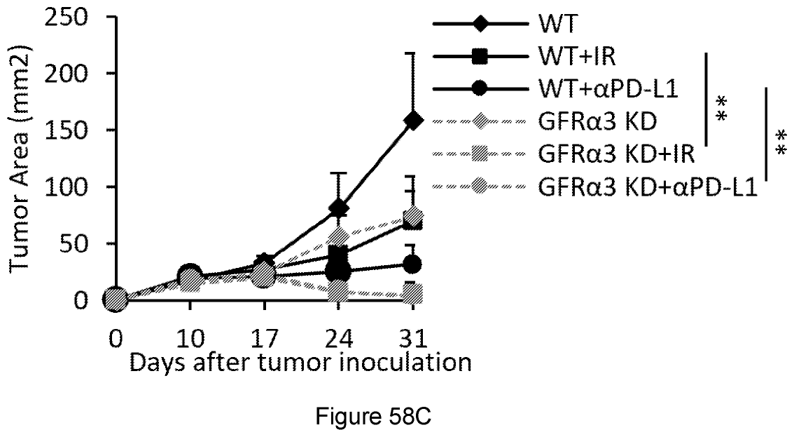

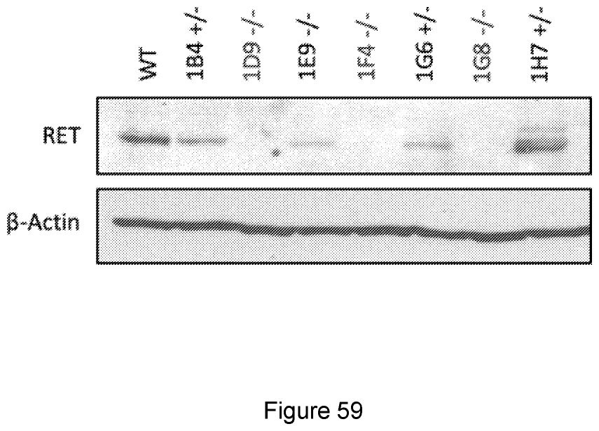

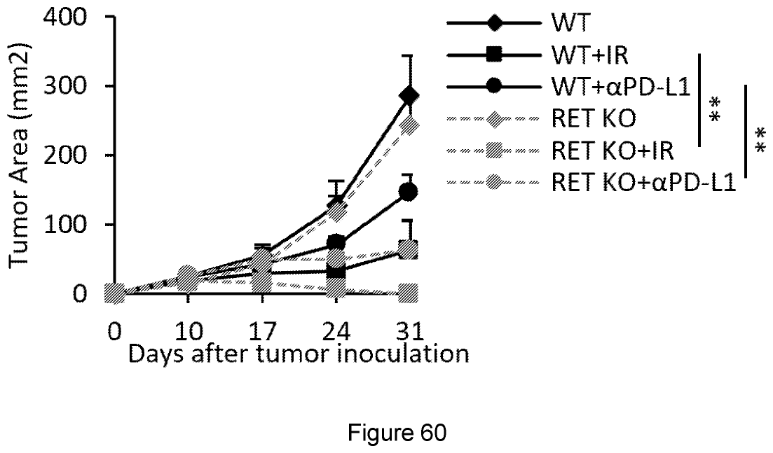

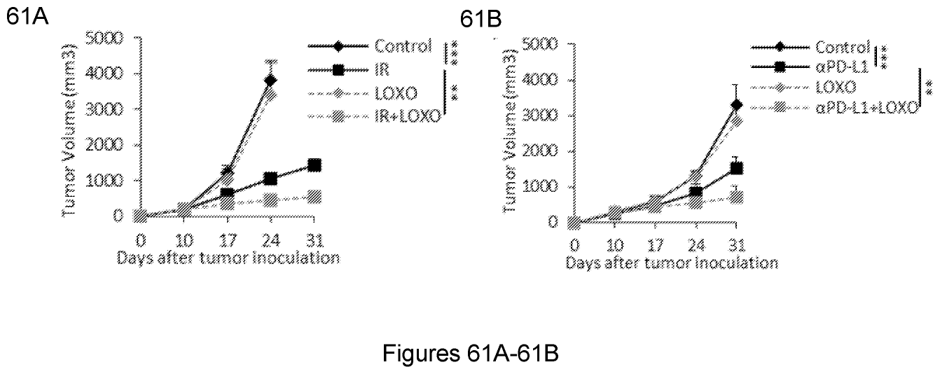

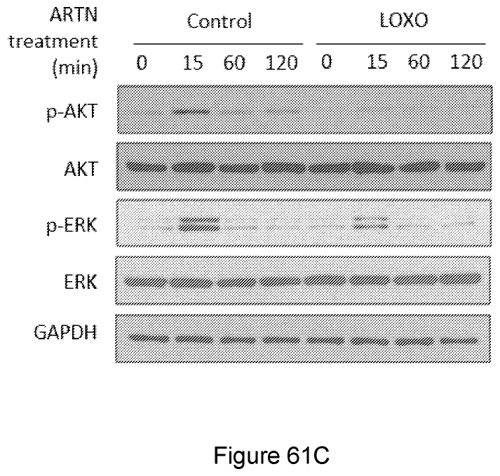

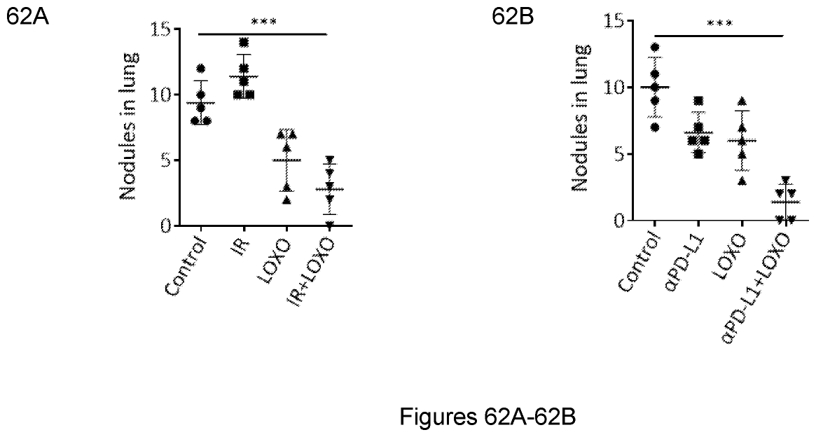

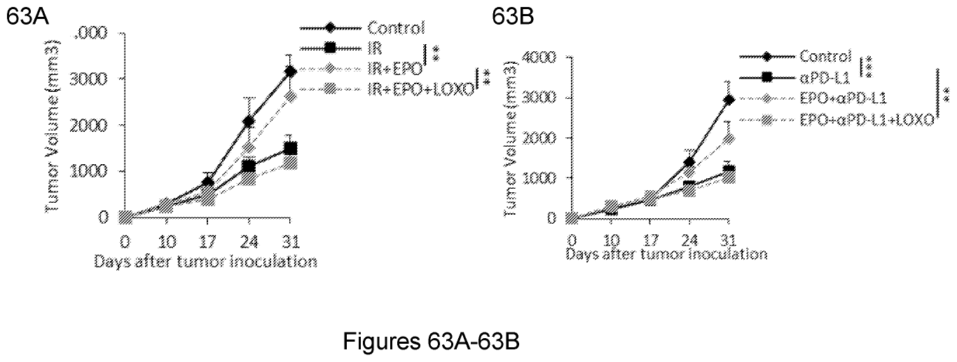

Next, whether blocking artemin enhances the anti-tumor treatment response was investigated. Intratumoral administration of artemin neutralizing antibody promoted the effect of IR and PD-L1 blockade (FIG. 58A). To identify the role of artemin signaling in cancer treatments, GFRα3 was knocked down in tumor cells (FIG. 58B) and better tumor control by either IR or anti-PD-L1 treatment (FIG. 58C) was observed compared with WT tumors. CRISPR/Cas9 genome editing was utilized to knock out RET, a co-receptor for artemin, in MC38 tumor cell lines (FIG. 59). RET knockout tumors showed enhanced therapeutic effects in the responses to IR and PD-L1 blockade treatments (FIG. 60), which suggests that artemin signaling in tumor cells played a critical role in the anti-tumor efficacies of radio- and immunotherapies. This finding also suggests that RET might be a potential target to enhance both radiotherapy and immunotherapies. To test this hypothesis, tumor-bearing mice were treated with either IR or anti-PD-L1 and LOXO-292, a RET selective inhibitor, and it was found that LOXO-292 enhanced the effects of IR and PD-L1 blockade (FIGS. 61A, 61B). In vitro treatment of tumor cells with LOXO-292 also inhibited artemin-induced activation of downstream signaling. These results indicate that artemin signals through RET in the tumor model (FIG. 61C). RET inhibition augmented the effects of both IR and PD-L1 blockade on spontaneous lung metastasis (FIGS. 62A, 62B). LOXO-292 also restored the therapeutic effects of IR and PD-L1 blockade that were initially impaired by EPO (FIGS. 63A, 63B), which suggests that the adverse effect of EPO on cancer therapy occurs through artemin/RET signaling. Thus, Ter-cell depletion, artemin neutralization, and inhibition of some of the artemin receptors all promoted the therapeutic effects of IR and PD-L1 blockade.

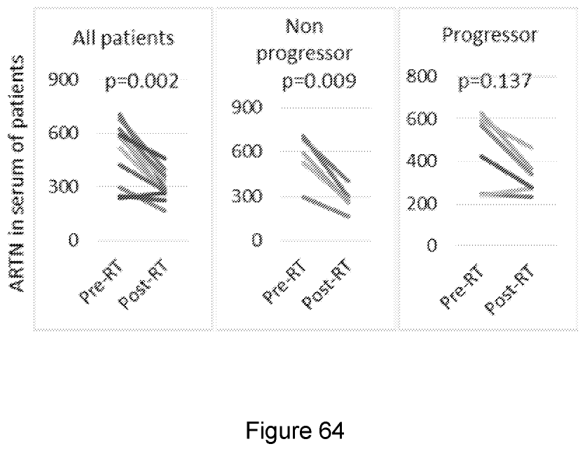

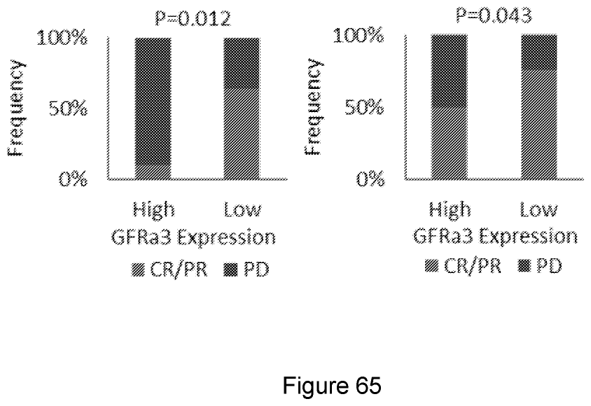

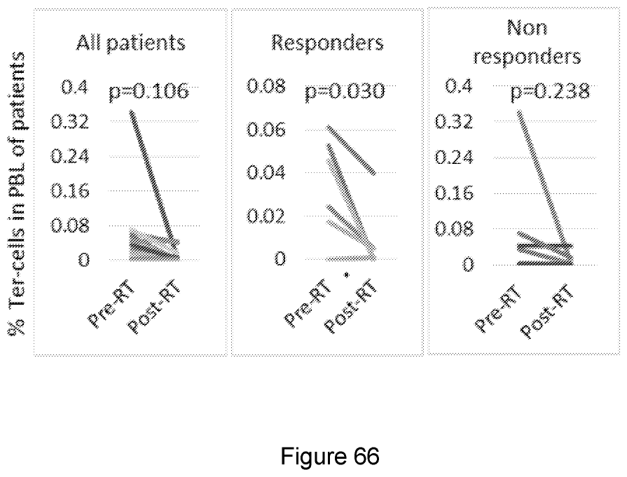

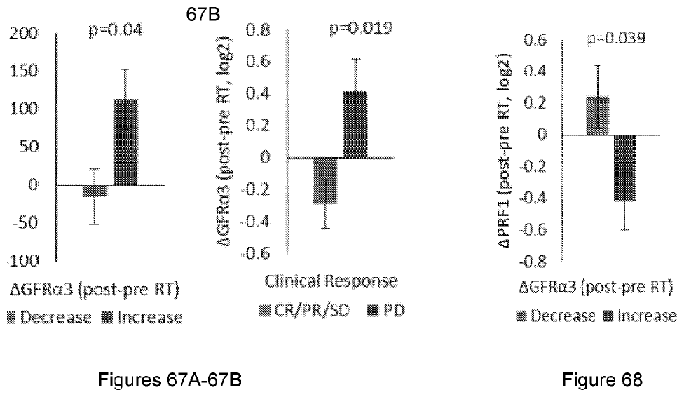

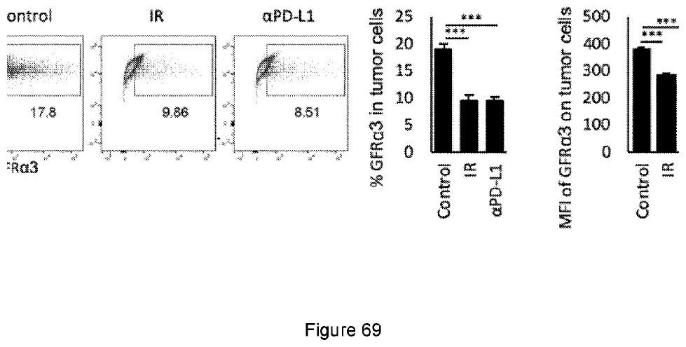

Radiation and immunotherapy responders exhibit treatment-induced Ter-cell reduction. Since artemin-secreting splenic Ter-cells are enriched in some cancer patients, and artemin levels correlated with poor prognosis in several different types of cancer patients, it was investigated whether differential responses to oncologic therapies were related to Ter-cell and artemin levels in cancer patients. Expression of one artemin receptor, GFRα3, was previously shown to be induced by artemin and correlated with serum artemin levels in cancer patients. Therefore, expression levels of GFRα3 were examined in a variety of human cancers from The Cancer Genome Atlas (TCGA), and it was found that GFRα3 was highly expressed in non-small cell lung cancer (NSCLC), colorectal cancer, and melanoma. In patients with NSCLC treated with chemoradiation therapy, it was found that the level of post-treatment circulating artemin protein decreased in those patients who had no evidence of disease recurrence following treatment, whereas artemin levels increased or were unchanged in those patients who developed disease recurrence (FIG. 64). In the context of immunotherapy, analysis of two clinical cohorts of patients with metastatic melanoma treated with anti-PD1+/−anti-CTLA-4 immunotherapy reported that high pretreatment GFRα3 expression was associated with a reduced probability of treatment response (complete or partial response vs. disease progression) compared with low GFRα3 expression (FIG. 65). In addition, in a clinical trial, patients treated with radiation therapy followed by pembrolizumab (anti-PD-1) immunotherapy (NCT02608385) were examined. It was determined that circulating Ter-cell abundance decreased post radiotherapy in patients with complete/partial responses to treatment but was unchanged in patients with disease progression in the response to radiation and immunotherapy (FIG. 66). In this cohort, tumor GFRα3 expression decreased post radiotherapy in treatment responders (FIGS. 67A, 67B). By contrast, GFRα3 expression increased in patients who progressed following treatment (FIGS. 68A, 68B). Additionally, decreased expression of tumor GFRα3 in response to RT was associated with increased expression of perforin 1 (PRF1), a marker of intratumoral cytolytic response (FIG. 69). To add to these results, in a murine tumor model, it was found that IR and PD-L1 blockade immunotherapy decreased the expression of GFRα3 on CD45− cells (FIG. 69), and GFRα3 knockdown tumors (FIG. 58B) responded significantly better to IR and PD-L1 blockade treatments compared with WT tumors. These data suggest that RT and immunotherapy responses are associated with decreases in Ter-cell and artemin levels.

### Conclusions

Findings. Local irradiation decreases tumor-induced Ter-cell accumulation in mouse spleen. IFNs and T cells are required for the effect of irradiation on Ter-cells. PD-L1 blockade reduces tumor-induced Ter-cell accumulation in a CD8+ T cell and IFN-γ-dependent manner. PD-L1 blockade reduces tumor-induced Ter-cells accumulation in spleen.

Ter-cells and artemin curtail the therapeutic effects of both RT and immunotherapy. Disrupting the Ter-artemin axis restored and enhanced the efficacy of both radiotherapy and anti-PD-L1 therapy. Suppression of the Ter/artemin axis is associated with response to RT and immune checkpoint blockade in cancer patients. GFRα3 knock down and RET knock out in tumor cells results in better tumor control by either IR or anti-PD-L1 treatment. RT and PD-L1 blockade reduces the expression of GFRα3 on tumor cells in vivo.

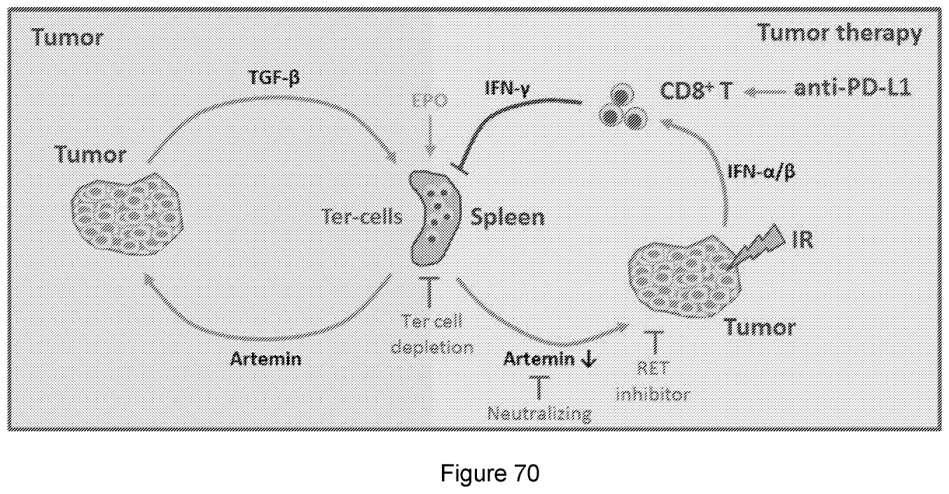

Further conclusions. These experiments demonstrated that radiotherapy and PD-L1 blockade reduced tumor-induced Ter-cells and artemin (FIG. 70). Blocking the Ter-cell/artemin axis promoted the therapeutic effects of both IR and anti-PD-L1 treatments. Increasing evidence demonstrates that radiation induces innate and adaptive immune responses mediated by IFNs, DCs, and CD8⁺ T cell responses, which are required for the full therapeutic effect of radiotherapy. This work describes for the first time an “abscopal” (out-of-field) effect of local tumor IR that suppresses Ter-cell accumulation in the spleen in a type I/II IFN-CD8⁺ T cell-dependent manner. Importantly, it was also found that PD-L1 blockade inhibited the production of Ter-cells in tumor-bearing mice through the actions of CD8⁺ T cells and IFN-γ. The finding that adoptive transfer of Ter-cells or administration of exogenous artemin or erythropoietin diminished the effects of both RT and PD-L1 blockade indicates that the reduction in the number of splenic Ter-cells contributed to restoration of the anti-tumor efficacy of IR and PD-L1 blockade.

These results demonstrate that IFN-γ is a necessary factor mediating Ter-cell death for the following reasons: 1) IR induced higher IFN-γ expression in splenic T cells; 2) IR did not induce high levels of apoptosis of Ter-cells in IFN-γ deficient mice compared with that of WT mice; and 3) intra-splenic injection of IFN-γ led to increased apoptosis of Ter-cells in the spleen of WT tumor-bearing mice. Increased abundance of T cells was also observed, as well as higher levels of MHC class I molecules on Ter-cells in spleens treated with IFN-γ. The mechanism may be direct induction of Ter-cell apoptosis and/or MHC I directed killing by T cells. Proinflammatory infections, including oncolytic virus therapy, which are able to increase IFN-γ production in spleen, are likely to inhibit Ter-cell accumulation and subsequently benefit tumor control. Increased ROS activity in CD45⁺Ter119⁺CD71⁺ EPCs has been described compared with CD45⁻Ter119⁺CD71⁺ Ter-cells, and it was reported that only the CD45⁻ EPCs exhibit overexpression of genes in the ROS pathway. In comparison, CD45⁻ Ter-cells have very low ROS levels; therefore, it is unlikely that ROS mediate Ter-cell apoptosis in these studies.

Although it was found that in mouse models using LLC and MC38 tumors, the spleen was the major (but not the exclusive) organ contributing to tumor-induced Ter-cell accumulation, it should be noted that the spleen is not a primary hematopoietic organ in humans except in certain disease states or stress conditions. For example, it was found that in mice, tumors increased Ter-cells in the liver, which decreased in response to IR and PD-L1 blockade, whereas bone marrow-derived Ter-cells showed no change in response to tumor inoculation or treatments (FIG. 6). Further investigation in humans is required to determine the origin(s) of artemin secreting Ter-cells during cancer development. Analysis of clinical trial samples indicated that Ter-cells in the peripheral blood of NSCLC patients decreased after radiotherapy treatment. The intratumoral expression of GFRα3, an artemin receptor, decreased in patients who responded to radiotherapy and/or PD-1 immunotherapy, but was unchanged in patients with poor responses to treatments.

Concerning the main contributor of artemin in tumor and blood, these findings mirrored those of other studies: artemin protein levels in the serum were significantly reduced after splenectomy in tumor-bearing mice. Other studies have found that in mice bearing artemin-knock-out tumors, splenic Ter-cell induction, serum artemin, and HCC growth were not significantly changed at the protein level, as compared with WT tumor controls. These results suggest tumor cells, which do express artemin, are not the main source of artemin in the serum. When tumors were irradiated or treated with anti-PD-L1, artemin protein levels in serum and total tumor homogenates decreased dramatically along with a reduction of Ter-cells, despite an artemin RNA increase in tumor cells. The results also indicate that artemin from the Ter-cells are the main contributor to artemin production in the serum and tumor microenvironment of tumor-bearing mice.

These findings have immediate clinical relevance. Here, it is reported that EPO administration increased Ter-cells in the spleen and liver of naive mice and abrogated IR- and anti-PD-L1-mediated reduction of Ter-cells and artemin, thereby blocking anti-tumor responses. Depletion of Ter-cells abrogated the adverse effects of EPO on the therapeutic efficacy of both IR and anti-PD-L1. Taken in the context of the other results, these findings suggest that unfavorable clinical outcomes following the administration of EPO and radiotherapy may be related to increases in Ter-cell abundance in cancer patients. Furthermore, EPO may exert indirect effects on T-cell functions via Ter-cells and their artemin production, as shown in FIG. 41A and FIG. 41B in which Ter-cells or artemin diminished T cells' tumor cell killing capacity. These findings implicate an indirect effect of EPO on cancer treatment outcomes, which differ from previous studies that focused on the direct action of EPO on tumor cells. Admittedly, these studies do not rule out the possibility that tumor cells under hypoxic conditions secrete EPO and contribute partially to the accumulation of Ter-cells in the spleen.

These results identify multiple strategies for targeting the Ter-cell/artemin axis to potentially improve the efficacy of both radiotherapy and immunotherapy, including Ter-cell depletion, artemin neutralization, and RET inhibition. An immediate potential translation is the use of selective RET inhibitors, including LOXO-292 and BLU-677, that have produced improved outcomes for patients with RET fusion-positive cancers. It was found that LOXO-292 promoted the effects of IR and PD-L1 blockade on both local tumor and spontaneous metastasis in a murine LLC model. Therefore, RET inhibitors might work as sensitizers to improve the efficacy of radiotherapy and immunotherapy by inhibiting RET tyrosine kinase activity driven by either gain-function mutations or a ligand of artemin secreted by tumor-induced Ter-cells.

### Example 2: Study of Artemin Pathway Modulation and Immune Cells

### Summary

The artemin pathway has a significant effect on immune cells. Artemin reduces CD8 T cell effector function, increases the Treg percentage in CD4 T cells, induces expression of PD-L1 on DCs and MDSCs, and induces GFRα3 expression on NK cells.

### Introduction

In light of the role of artemin in cancer treatment, further exploration of the effects of artemin pathway modulation on immune cells, including T cells, was warranted. The studies performed helped to elucidate the role played by artemin in creating an environment that suppresses T cells. Furthermore, the results demonstrate that the artemin pathway acts through multiple downstream binding partners.

### Methods

In vitro testing: CD8 T cell treatment with artemin. CD8 T cells were isolated from spleens of naïve mice. CD8 T cells were cultured with T cell activation beads for 3 days and labeled with DNA dye cellTrace violet, followed by co-culturing with 150 ng/mL recombinant artemin for 24 hours. T cells were then washed and stimulated by stimulation cocktail and protein transport cocktail for 6 hours before being subjected to intracellular antibody staining.

In vivo testing: MC38 murine colon cancer treatment with artemin. Established MC3 8 murine colon cancer tumors were treated with artemin by intra-tumoral injection. Tumors were digested into single cell suspension, stimulated and stained with intracellular antibodies. Cells were analyzed by flow cytometry.

Quantitative PCR (qPCR). Expression of possible artemin receptors GFRα1 (primers: SEQ ID NOs: 5, 6), GFRα3 (primers: SEQ ID NOs: 7, 8), Syndecan 3 (primers: SEQ ID NOs: 9, 10), NCAM (primers: SEQ ID Nos: 11, 12), and RET (SEQ ID Nos: 13, 14) was analyzed by qPCR in CD8 T cells and bone marrow derived MDSCs during co-culture with artemin.

Radiation (IR) treatment analysis. We treated established Lewis lung carcinoma (LLC) tumors with control, artemin, IR, and artemin+IR (20 Gy IR). Flow cytometry with staining of GFRα1, GFRα3 and NCAM receptors was performed.

### Results

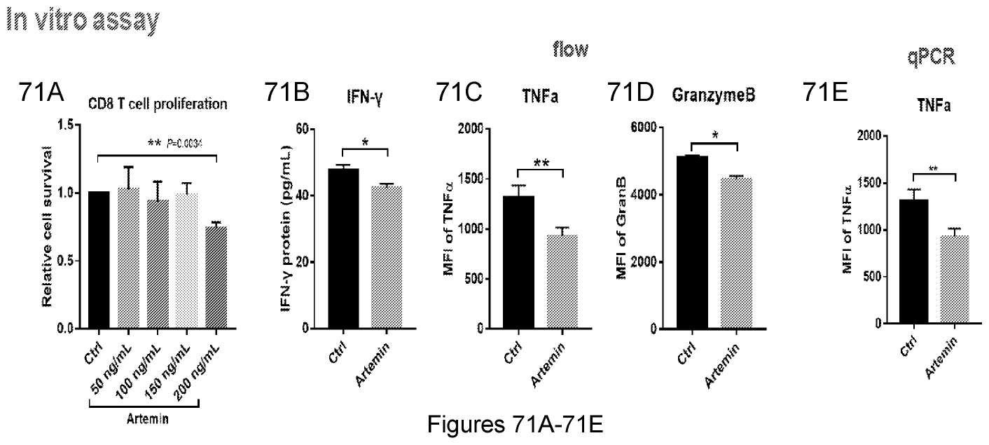

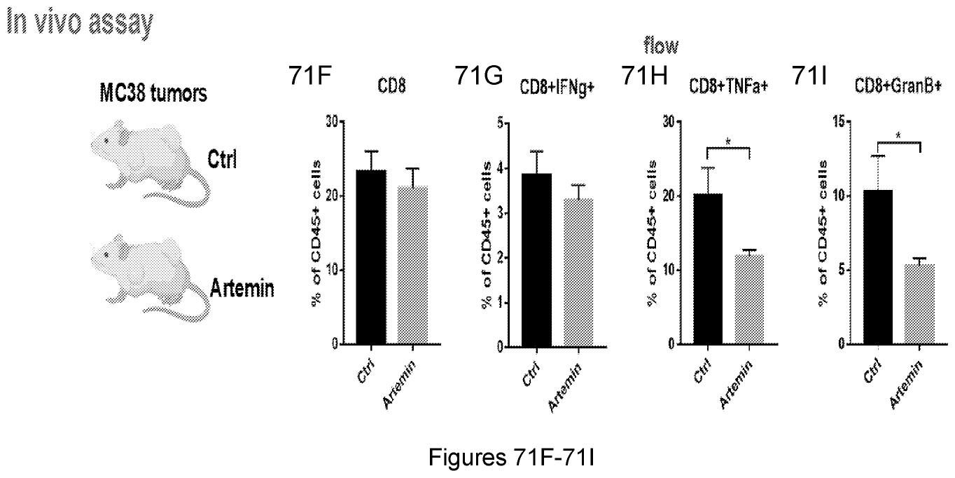

Artemin directly inhibits T cell proliferation and attenuates T cell effector function in vitro at 200 ng/mL. Artemin treatment at 200 ng/mL resulted in reduced CD8 T cell proliferation (FIG. 71A). Furthermore, artemin treatment also led to decreases in production of IFN-γ, granzyme B, and tumour necrosis factor alpha (TNFα) in T cells at the protein level (FIGS. 71B-71D) and RNA level (FIG. 71E).

Artemin affects T cell function in vivo. Artemin administration did not alter frequency of total CD8 T cell among CD45+ cells (FIG. 71F). However, for molecules indicative of effector functions, while changes in IFN-γ production were not significant (FIG. 71G), production of granzyme B and TNFα proteins were both significantly reduced (FIGS. 71H-71I).

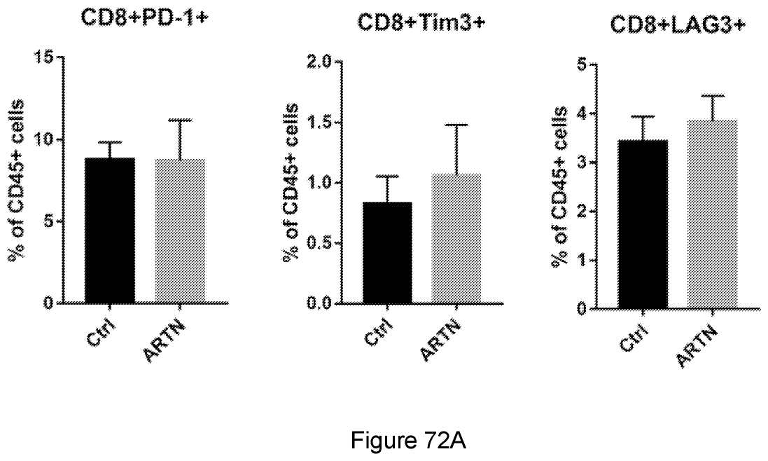

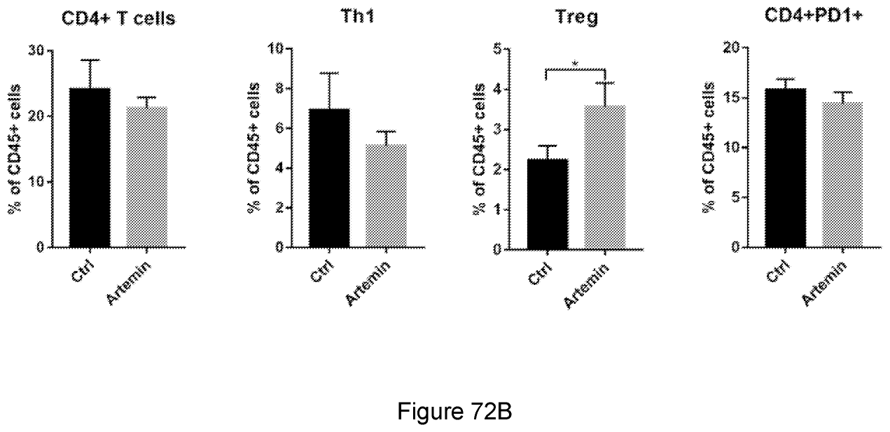

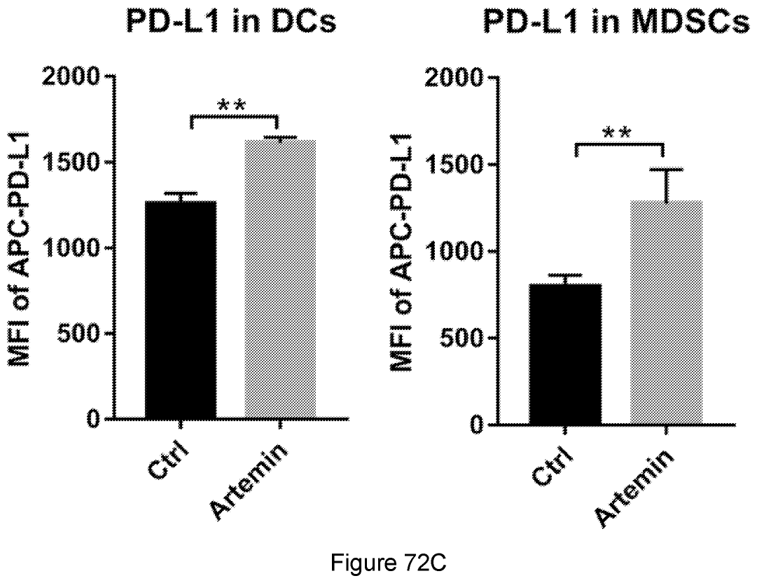

Artemin does not affect exhaustion status of intratumoral T cells. Artemin treatment did not change the expression of exhaustion markers PD-1, LAG3, and T cell immunoglobulin and mucin domain-containing protein 3 (TIM3) (FIG. 72A).

Artemin increases Treg percentage in CD4 T cells. While CD4 T helper cell numbers were not significantly changed, Treg percentage in CD4 T cells was significantly increased. (FIG. 72B).

Artemin can induce expression of PD-L1 on some immune cell types. Artemin induced PD-L1 expression on dendritic cells (CD11C+) and MDSCs (CD11b+Ly6C+) (FIG. 72C).

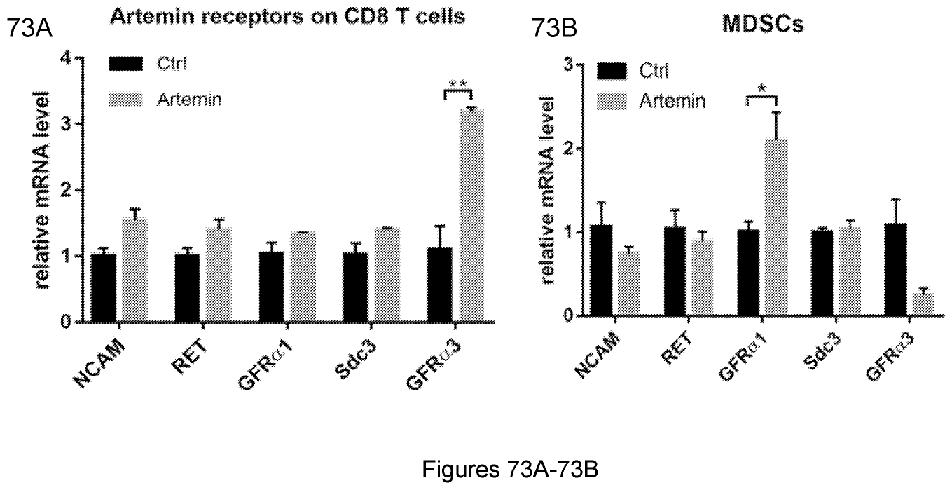

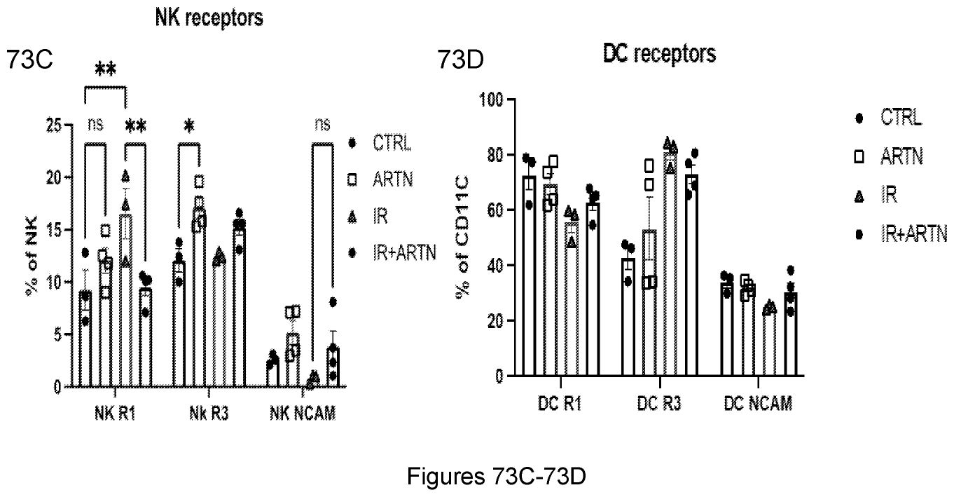

Artemin can induce expression of multiple receptors. Induction of GFRα3 was observed in CD8 T cells, and induction of GFRα1 was observed in MDSC cells (FIGS. 73A-73B). Radiation induced expression of GFRα1 on NK cells and GFRα3 on DC cells whereas artemin induces GFRα3 expression on NK cells (FIGS. 73C-73D).

### Conclusions

Findings. Artemin directly attenuates CD8 T cell proliferation and effector function in vitro and in vivo. Furthermore, artemin upregulates PD-L1 expression in DCs and MDSCs in MC38 tumors. Artemin upregulates GFRα3 in CD8 T cells and GFRα1 in MDSC cells.

Further conclusions. Artemin diminishes cytotoxic effector function of T cells in both in vitro and in vivo studies. While artemin may not be able to deepen the exhaustion state of T cells, it can result in a more suppressive tumor microenvironment which contributes to inhibiting T cell effector function. As other family members in GNDF family do, artemin may rely on distinct partners to transduce signals in different cell types responding to different environment stimuli. Therefore, compared to downstream approaches, blocking artemin signaling might be a more efficient and focused method to alleviate artemin-mediated inhibition of anti-tumor immunity.

SEQUENCES

| SEQ | | |
|---|---|---|
| ID | | |
| NO: | Name | Sequence |
| 1 | artemin upstream | TACTGCATTGTCCCACTGCCTCC |
| | primer | |
| 2 | artemin downstream | TCGCAGGGTTCTTTCGCTGCACA |
| | primer | |
| 3 | GAPDH upstream | AGACCAGCCTGAGCAAAAGA |
| | primer | |
| 4 | GAPDH downstream | CTAGGCTGGAGTGCAGTGGT |
| | primer | |
| 5 | mGFRa1_q_F | CACTCCTGGATTTGCTGATGT |
| 6 | mGFRa1_q_R | AGTGTGCGGTACTTGGTGC |
| 7 | mGFRa3_q_F | AGAGAACAGGTTTGTGAACAGC |
| 8 | mGFRa3_q_R | CAGCGGCCTGCTTAAACTG |
| 9 | mSdc3_q_F | AGAGGCCGGTGGATCTTGA |
| 10 | mSdc3_q_R | CTCCTGCTCGAAGTAGCCAGA |
| 11 | mNCAM_q_F | ACCACCGTCACCACTAACTCT |
| 12 | mNCAM_q_R | TGGGGCAATACTGGAGGTCA |
| 13 | mRET_q_F | TCTATGGCGTCTACCGTACAC |
| 14 | mRET_q_R | GGGAAACCACCATTGCGGAT |
| 5 | mGFRa1_q_F | CACTCCTGGATTTGCTGATGT |
| 6 | mGFRa1_q_R | AGTGTGCGGTACTTGGTGC |
| 7 | mGFRa3_q_F | AGAGAACAGGTTTGTGAACAGC |
| 8 | mGFRa3_q_R | CAGCGGCCTGCTTAAACTG |
| 9 | mSdc3_q_F | AGAGGCCGGTGGATCTTGA |
| 10 | mSdc3_q_R | CTCCTGCTCGAAGTAGCCAGA |
| 11 | mNCAM_q_F | ACCACCGTCACCACTAACTCT |
| 12 | mNCAM_q_R | TGGGGCAATACTGGAGGTCA |
| 13 | mRET_q_F | TCTATGGCGTCTACCGTACAC |
| 14 | mRET_q_R | GGGAAACCACCATTGCGGAT |

What is claimed is:

1. A method of treating cancer in a subject, comprising:
a) administering to the subject an effective amount of at least one of a radiotherapy and a checkpoint inhibitor; and
b) administering to the subject an effective amount of an inhibitor of the artemin pathway.

2. The method of claim 1, wherein the method comprises administering a radiotherapy.

3. The method of claim 1, wherein the method comprises administering a checkpoint inhibitor.

4. The method of claim 1, wherein the method comprises administering a radiotherapy and a checkpoint inhibitor.

5. The method of claim 1, 3, or 4, wherein the checkpoint inhibitor is an antibody or antigen-binding fragment thereof.

6. The method of claim 1, 3, or 4, wherein the checkpoint inhibitor is a peptide.

7. The method of claim 1, 3, or 4, wherein the checkpoint inhibitor is a small molecule.

8. The method of claim 1, 3, or 4, wherein the checkpoint inhibitor inhibits PD-L1.

9. The method of claim 8, wherein the checkpoint inhibitor is an anti-PD-L1 antibody or antigen-binding fragment thereof.

10. The method of claim 8, wherein the checkpoint inhibitor is a peptide.

11. The method of claim 8, wherein the checkpoint inhibitor is a small molecule.

12. The method of any of claims 1-4, wherein the inhibitor of the artemin pathway is an antibody or an antigen-binding fragment thereof.

13. The method of any of claims 1-4, wherein the inhibitor of the artemin pathway is a small molecule.

14. The method of any of claims 1-4, wherein the inhibitor of the artemin pathway is a gene editing composition.

15. The method of claim 14, wherein the gene editing composition comprises CRISPR/Cas9.

16. The method of claim 14, wherein the gene editing composition inhibits RET or GFRα3.

17. The method of any of claims 1-4, wherein the inhibitor of the artemin pathway inhibits artemin.

18. The method of claim 17, wherein the artemin inhibitor is an anti-artemin antibody or antigen-binding fragment thereof.

19. The method of any of claims 1-4, wherein the inhibitor of the artemin pathway inhibits GFRα3.

20. The method of any of claims 1-4, wherein the inhibitor of the artemin pathway inhibits RET.

21. The method of claim 20, wherein the RET inhibitor is one or more of vandetanib, cabozantinib, RXDX-105, lenvatinib, sorafenib, sunitinib, dovitinib, alectinib, ponatinib, regorafenib, nintedanib, apatinib, motesanib, BLU-667, or LOXO-292.

22. The method of claim 21, wherein the RET inhibitor is LOXO-292.

23. The method of any of claims 1-4, wherein the cancer is lung cancer, colon cancer, or melanoma.

24. The method of claim 1, 3, or 4, wherein the checkpoint inhibitor and the inhibitor of the artemin pathway are in the same composition.

25. The method of claim 1 or 4, wherein the checkpoint inhibitor and/or the inhibitor of the artemin pathway are administered subsequent to the radiotherapy.

26. The method of claim 25, wherein the checkpoint inhibitor and/or the inhibitor of the artemin pathway are administered 3-10 days subsequent to the start of the administration of the radiotherapy.

27. The method of any of claims 1-4, wherein the inhibitor of the artemin pathway is administered simultaneously with the checkpoint inhibitor and/or the radiotherapy.

28. The method of any of claims 1-4, wherein the inhibitor of the artemin pathway is administered subsequent to the checkpoint inhibitor and/or the radiotherapy.

29. The method of claim 28, wherein the inhibitor of the artemin pathway is administered no more than 7 days subsequent to the checkpoint inhibitor and/or the radiotherapy.

30. The method of any of claim 1, 3, or 4, wherein the checkpoint inhibitor is administered to the subject at more than one time.

31. The method of claim 30, wherein the checkpoint inhibitor is administered every other week.

32. The method of claim 31, wherein the checkpoint inhibitor is administered simultaneously with the radiotherapy.

33. The method of claim 31, wherein the checkpoint inhibitor is administered subsequent to the radiotherapy.

34. The method of any of claims 1-4, wherein the inhibitor of the artemin pathway is administered to the subject at more than one time.

35. The method of claim 34, wherein the inhibitor of the artemin pathway is administered every other day.

36. The method of claim 35, wherein the inhibitor of the artemin pathway is administered for 14 days simultaneously with and subsequent to radiotherapy.

37. The method of claim 34, wherein the inhibitor of the artemin pathway is administered every day.

38. The method of claim 37, wherein the inhibitor of the artemin pathway is administered until remission is achieved.

39. The method of any of claim 1, 3, or 4, wherein the checkpoint inhibitor is administered intravenously.

40. The method of any of claims 1-4, wherein the inhibitor of the artemin pathway is administered intratumorally.

41. The method of any of claims 1-4, further comprising c) reducing the size of a tumor or inhibiting growth of a tumor in the subject.

42. A method of treating cancer in a subject, comprising:
a) administering to the subject an effective amount of ionizing radiation, an effective amount of an anti-PD-L1 antibody or an antigen-binding fragment thereof, and an effective dose of an anti-artemin antibody or an antigen-binding fragment thereof; and
b) reducing the size of a tumor or inhibiting growth of a tumor in the subject.

43. A method of treating cancer in a subject, comprising:
a) administering to the subject an effective amount of ionizing radiation and an effective amount of LOXO-292; and
b) reducing the size of a tumor or inhibiting growth of a tumor in the subject.

44. A method of treating cancer in a subject, comprising:
a) administering to the subject an effective amount of an anti-PD-L1 antibody or an antigen-binding fragment thereof and an effective amount of LOXO-292; and
b) reducing the size of a tumor or inhibiting growth of a tumor in the subject.

45. A composition, comprising:
a) an effective amount of a checkpoint inhibitor;
b) an effective amount of an inhibitor of the artemin pathway; and
c) a pharmaceutically acceptable carrier, solvent, adjuvant, diluent, or combination thereof.

46. The composition of claim 45, wherein the checkpoint inhibitor is an antibody or an antigen-binding fragment thereof.

47. The composition of claim 45, wherein the checkpoint inhibitor is a peptide.

48. The composition of claim 45, wherein the checkpoint inhibitor is a small molecule.

49. The composition of claim 45, wherein the inhibitor of the artemin pathway is an antibody or an antigen-binding fragment thereof.

50. The composition of claim 45, wherein the inhibitor of the artemin pathway is a small molecule.

51. The composition of claim 45, wherein the inhibitor of the artemin pathway is a gene editing composition.

52. The composition of claim 45, wherein the checkpoint inhibitor is a PD-L1 inhibitor.

53. The composition of claim 45, wherein the inhibitor of the artemin pathway is LOXO-292.

54. A composition, comprising:
a) an effective amount of an anti-PD-L1 antibody or an antigen-binding fragment thereof;
b) an effective amount of an anti-artemin antibody or an antigen-binding fragment thereof; and
c) a pharmaceutically acceptable carrier, solvent, adjuvant, diluent, or combination thereof.

55. A composition, comprising:
a) an effective amount of an anti-PD-L1 antibody or an antigen-binding fragment;
b) an effective amount of LOXO-292; and
c) a pharmaceutically acceptable carrier, solvent, adjuvant, diluent, or combination thereof.
